(19) United States
(12) Patent Application Publication
(10) Pub. No.: US 2022/0347180 A1
(43) Pub. Date: Nov. 3, 2022

(54) ENHANCING CANCER THERAPY TREATMENT WITH BH3 MIMETICS

(71) Applicant: THE REGENTS OF THE UNIVERSITY OF COLORADO, A BODY CORPORATE, Denver, CO (US)

(72) Inventors: Yiqun Shellman, Aurora, CO (US); Richard Tobin, Aurora, CO (US); Nabanita Mukherjee, Aurora, CO (US); David Norris, Englewood, CO (US); Martin D.` Carter, Golden, CO (US)

(21) Appl. No.: 17/764,043

(22) PCT Filed: Sep. 25, 2020
(86) PCT No.: PCT/US20/52790

§ 371 Date: Mar. 25, 2022

Related U.S. Application Data
(60) Provisional application No. 62/907,179, filed on Sep. 27, 2019.
(60) Provisional application No. 63/056,324, filed on Jul. 24, 2020.

(51) Int. Cl. A61K 31/519; A61K 39/395; A61K 31/635; A61P 35/00

(52) U.S. Cl. CPC A61K 31/519; A61K 39/3955; A61K 31/635; A61P 35/00

(57) ABSTRACT

Disclosed herein are methods for using therapeutic compounds, for example BH3 mimetics such as MCL1 inhibitors, to treat patients that may have failed to respond to targeted or immunotherapy treatments. In several embodiments, methods for treating such patients include combining MCL1 inhibitors with immunotherapies to enhance immunotherapy treatment. For example, the combination of MCL1 inhibitors with immunotherapies is used to treat cancer, such as melanoma. In some embodiments, MCL1 inhibitory compounds and therapies may be useful in treating tissues or tumors having one or more MDSC cell expressing high levels of MCL1. In some embodiments, MCL1 and BCL2 inhibitory compounds and therapies comprising same may be used to treat one or more melanomas, for example cutaneous melanoma, melanoma subtypes selected from uveal, acral, and mucosal. In some embodiments, MCL1 inhibitory compounds and therapies comprising same may be used to treat one or more melanomas, for example uveal melanoma. In many embodiments the targeted cells or tissues may not have BRAF mutations.

### CROSS-REFERENCE TO RELATED APPLICATIONS

This application claims benefit of priority pursuant to 35 U.S.C. § 119(e) of U.S. provisional patent applications No. 63/056,324, filed on 24 Jul. 2020, and 62/907,179 filed 27 Sep. 2019, both of which are hereby incorporated by reference in their entireties.

### FIELD

The disclosed processes and methods are generally directed to using BH3 mimetics to enhance cancer therapies, and more specifically to enhance anti-tumor therapies.

### BACKGROUND

Melanoma is a significant and rising health burden throughout the United States. Melanoma is indiscriminate of age and sex and is the second most common form of cancer among patients aged 15-29. Current estimates predict that more than 178,000 Americans will be diagnosed with and more than 10,000 people will die of melanoma in 2019. Despite recent improvements in treating melanoma patients, over-all survival rates remain low among patients with advanced stage disease.

Melanomas have many subtypes. Cutaneous melanomas are the most common form and derived from pigment-producing cells (melanocytes) in the skin, with a high frequency of mutations at BRAF-V600. Rare subtypes of melanomas include uveal (eye melanomas), mucosal (melanoma from the tissues that line internal areas of the body), and acral (melanomas occurring on the hands and feet). Many of these rare subtypes of melanomas are genetically distinct from cutaneous melanomas and typically lack BRAF-V600E/K mutations.

The incidence of invasive melanoma cases has increased by 54% in the last decade. Treatment of advanced melanoma has dramatically improved in recent years, and currently include targeted therapies against BRAF or MEK, and immunotherapy. Targeted therapies work only on a subset of patients with specific mutations of BRAF-V600E/K; however, of patients that initially respond, almost all relapse. Although immunotherapies are promising, not all patients respond and some patients also relapse. Furthermore, many of the rare subtypes of melanoma currently do not have any FDA-approved drugs for treatments, and a large proportion of these patients do not respond to immunotherapies. Thus, options are still limited for patients without mutations suitable for targeted therapies and for those that do not respond to immunotherapies, or relapse to treatment.

Despite advances in the treatment of cancer, such as melanoma, a significant number of patients do not respond to or become resistant to current therapies. For example, a patient with a suppressed immune system may not respond to immunotherapies designed to elicit an immune response to tumor cells. In other words, treatments with immunotherapies alone are ineffective at eliminating tumors.

Melanomas can also occur in pets. For example, mucosal melanomas are frequent in dogs, where BRAF is rarely mutated. In addition, the high cost of immunotherapies makes these therapies unlikely options for pets. Thus, there are no good options other than surgical removal to treat melanoma in pets.

The information included in this Background section of the specification, including any references cited herein and any description or discussion thereof, is included for technical reference purposes only and is not to be regarded subject matter by which the scope of the invention as defined in the claims is to be bound.

### SUMMARY

Disclosed herein are methods of using BH3 mimetics to treat patients resistant to immunotherapy and patients who do not benefit from existing treatments.

In one embodiment, a method for enhancing immune function in a patient in need thereof is disclosed. The method may include administering to the patient a therapeutically effective dose of a first therapeutic composition comprising at least one immunoglobulin; administering to the patient a therapeutically effective dose of a second therapeutic compound comprising a molecule with affinity for BCL2; allowing the second therapeutic compound to reduce a size or activity of a population of immunosuppressive and increase a size or activity of a population of immunostimulatory cells; and thereby enhancing immune function in the patient. The first therapeutic composition may be a checkpoint inhibitor. The first population of immunosuppressive cells may include one or more myeloid-derived suppressor cells. The second population of immunostimulatory cells may include one or more CD8+ T cells. The patient may be a mammal or human. In embodiments where the patient is a human, the human may have a condition selected from a solid tumor, melanoma, virally-induced cancer, or viral infection.

In another embodiment, a method of reducing or eliminating solid tumors is disclosed, the method may include administering a therapeutic dose of a BCL2 family member inhibitor to inhibit immunosuppression of T cells; administering an immunotherapeutic compound to increase immune activation within the solid tumor and induce solid tumor reduction and/or elimination, in some cases, inhibitors against the pro-survival BCL2 family members may enhance the efficacy of the immunotherapeutic compound, and may be an MCL1 inhibitor, that may inhibit immunosuppression of CD8+ T cells to facilitate reducing or eliminating solid tumors, for example the solid tumor may be melanoma. In some embodiments, the BCL2 family member inhibitor may inhibit immunosuppression of T cells by targeting myeloid-derived suppressor cells.

In yet another embodiment, a method of improving vaccine function is disclosed, the method may include introducing a therapeutically effective dose of a BH3 mimetic to a patient in need of vaccination treatment to reduce or eliminate suppression of the patient's immune system; vaccinating the patient with a vaccine treatment to produce antibodies and/or T cells having improved immune function due to the therapeutic effect of the BH3 mimetic, in some cases, the BH3 mimetic may improve the immune system response to the vaccine treatment.

In still another embodiment, a method of treating a subject resistant to or relapsed from an immunotherapy is disclosed, the method may include administering a therapeutic compound targeting pro-survival BCL-2 family members to inhibit immunosuppression of T cells; administering an immunotherapy to increase T cell recognition of tumor cells, in some cases, the immunotherapy may block the interaction of checkpoint proteins interfering with T cell recognition of tumor cells. The immunotherapy when administered by itself, without the therapeutic compound, may be insufficient to affect tumor cells. In some embodiments, the therapeutic compound may be an MCL1 inhibitor. The immunotherapy may be anti-PD1. In any of the embodiments, the therapeutic compound with affinity for BCL2 family members, may be one or more of a MCL1 inhibitor, BH3 mimetic, S63845, or S64315.

In another embodiment, a method for treating melanoma in a patient in need thereof is disclosed, the method may include administering to the patient a therapeutically effective dose of a first therapeutic composition comprising a first BH3 mimetic compound; administering to the patient a therapeutically effective dose of a second therapeutic composition comprising a second BH3 mimetic compound; allowing the first and second therapeutic compounds to act synergistically to induce death of melanoma cells; and thereby effectively treating advanced melanoma in the patient. In some embodiments, the first therapeutic composition may include a molecule with BCL2 inhibitory activity. The first therapeutic composition may include ABT-199/venetoclax. The second therapeutic composition may be a molecule with MCL1 inhibitory activity, for example the second therapeutic composition may include S63845 or S64315/MIK665. The melanoma may not include BRAF-V600E/K mutations, and the induction of death may be via programmed cell death. The patient may be a mammal, a human, or a pet.

In yet another embodiment, a method for treating a melanoma cell is disclosed, the method may include contacting the cell with a first BH3 mimetic compound; contacting the cell with a second BH3 mimetic compound; thereby treating the cell. The first BH3 mimetic compound may be a molecule with MCL1 inhibitory activity, for example, the first BH3 mimetic compound may be S63845 or S64315/MIK665.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter. A more extensive presentation of features, details, utilities, and advantages of the present invention as defined in the claims is provided in the following written description of various embodiments and implementations and illustrated in the accompanying drawings.

### BRIEF DESCRIPTION OF THE DRAWINGS

FIG. 1 is an image illustrating exemplary pathways over which MCL-1 inhibitors enhance anti-tumor immunity.

FIG. 2 shows result from various in-vitro and in vivo studies, demonstrating MCL1 inhibitor's ability to increase antitumor immunity. Panel A shows a graph illustrating cell viability of cells treated with MCL-1i in vitro. Panel B shows a graph illustrating tumor growth over time for tumors of B16 melanoma in C57BL/6 syngeneic mouse model, treated with MCL-1i. Panel C shows graphs illustrating flow cytometry results for tumor-infiltrating immune cells comparing vehicle control tumors and MCL-1i-treated tumors, harvested from those shown in Panel B.

FIG. 3 shows a graph comparing tumor growth over time for tumors of the similar model as in FIG. 2B, treated with MCL-1i alone, anti-PD1 alone, and a combination of the two. Results demonstrated MCL1 inhibitors increased efficacy of anti-PD1 treatment.

FIG. 4 is an exemplary plan for studying the presently disclosed compounds. Panel A is a table illustrating exemplary experimental groups, and Panel B is an image illustrating an exemplary treatment schedule for administering MCL-1i and anti-PD1.

FIG. 5 shows an exemplary gating strategy to identify tumor-infiltrating immune cells. The inset table shows markers to identify tumor-infiltrating immune cells.

FIG. 6 shows differential expression of apoptotic-related mRNA and proteins in the TCGA cutaneous melanoma dataset and the effects of knocking down BCL2 or MCL1. (a and b) Comparison between two genotypic groups: BRAF-hotspot mutated (MUT) (n=110) and BRAF-WT (WT) (harboring RAS hotspot mutated, any NF1 mutated, and triple wild type n=122). Panel (a) mRNA expression values for BCL2, CASP8, PDCD4, and MCL1. Panel (b) Relative RPPA protein expression values for PDCD4, CASP8, and BCL2. MCL1 was not included on the RPPA panel. Each dot represents an individual sample, and the horizontal line represents the mean. Panels (c) and (d) show the effects of BCL2 or MCL1 knockdown in A375 cells. Cells were treated with the indicated drugs for 48 h. Knocking down BCL2 (shBCL2) sensitized cells to MCL1 inhibitor S63845 and knocking down MCL1 (shMCL1) sensitized cells to BCL2 inhibitor ABT-199. Y-axis shows percentage of relative viability and X-axis indicates the BH3 mimetics used. ** indicates p<0.01; *** indicates p<0.001. Error bars represent +/−SEM. (e) shows the immunoblots confirming the knockdown.

FIG. 7 shows the combination of the BCL2 inhibitor ABT-199 with the MCL1 inhibitor S63845 had high efficacy in advanced melanomas in vitro. Panel (a) ATP assays for various subtypes of melanoma patient samples and normal human melanocytes upon indicated treatments for 48 h. The viability of the DMSO control for each cell line was set to 100%. Panel (b) shows higher efficacy for BRAF-WT melanomas. Summary of ATP assay data of fifteen melanoma cell lines and patient samples and one primary melanocyte line treated with vehicle, single drug or combination of S63845+ABT-199 at a dose of 2.5 μM. Black dotted line indicate 50% relative viability. Panel (c) Dot plot of IC50 values for combination treatment in BRAF-V600E-MUT and BRAF-WT lines. Each dot represents one cell line. Panel (d) Dot plot with the Combination-Index (CI) of the drug combination at 2.5 μM dose calculated using Compusyn software (version 1). CI values <0.5 (red line) indicate very strong synergism. Smaller CI values indicate stronger synergy. For visual clarity, the * is not shown in panels a and b. Panel (e) Immunoblot with lysates collected after 48 h treatment with DMSO, single drugs, or combination, and probed for PARP. Both combinations increased the cleaved product of PARP. Molecular weight markers are in kDa. Panel (f) shows S63845 combined with ABT-199 induced apoptosis in melanoma cells. Phase-contrast images melanoma cells showing cell death in combination treatment after 48 h. The images in are representatives of the cells collected for immunoblot analysis shown in FIG. 7e. Scale bar=100 μm.

FIG. 8 shows the treatment of ABT-199 plus S63845 significantly inhibited tumor growth without affecting mouse weight. A BRAF-WT line, MB3616, was used in the xenograft study, and tumor volume (Panel a) and weight of the mice (Panel b) were measured. The combination's inhibiting effects on tumor volume was statistically significant, compared to vehicle or the single drugs (p<0.001) Panel (a). Panel (c) shows representative bright-field images of Ki67 and Cleaved Caspase-3 staining from tumor sections derived from mouse xenografts experiments. Scale bar, 50 μm. The summary quantifications are in Panel (d) for Ki67 and Panel (e) for cleaved Caspase 3 positive area. The effects of the combination were statically significant, compared to vehicle or individual treatments, and we only show the least significant p-value of the comparisons. * indicates p<0.05; *** indicates p<0.001. Error bars represent +/−SEM. Panels (f) and (g) show combination of S63845+ABT-199 kills BRAF-WT melanoma cell in vitro at sub-micromolar dose. Panel (f) shows a summary of ATP assay data of melanoma cell lines and patient samples treated with vehicle, single drug or combination of S63845+ABT-199 at a dose of 0.625 uM. Panel (g) Dot plot with the Combination-Index (CI) of the drug combination at 0.625 μM dose calculated using Compusyn software (version 1). CI values<0.5 (red line) indicate very strong synergism. Smaller CI values indicate stronger synergy. Panels h and i show combination of S63845+ABT-199 kills BRAF-VG00E melanoma cells in vivo at higher frequency of treatment. Panel (h) Tumor volume in mouse xenograft models with BRAF-V600E A375 cells. The combination treatment significantly inhibited the tumor growth compared to vehicle or the single drugs. For visual clarity, we marked only the combinational treatment that was significantly different from comparisons with the vehicle and the single drug treatments. Within each significant combination treatment, we only show the least significant p-value of the comparisons * indicates p<0.05; Panel (i) Weight of the mice during the treatment period of the experiment from Panel (h). Error bars represent +/−SEM for all panels. Panel (j) shows endogenous level of BID in melanoma cell lines and patient samples. Immuno blot to show the endogenous level of BID in representative BRAF MUT and WT cell lines and patient samples.

FIG. 9 shows the combination of ABT-199 with S63845 significantly inhibited sphere-forming capacity of the Melanoma Initiating Cells. Melanoma cells were subjected to the primary sphere assay Panels (a-c). Spheres were treated with the indicated compounds either alone, or in combination, for 48 h, and the number of surviving spheres were counted and quantified Panel (a), and Panel (b) shows example images by phase contrast microscopy. Panel (c) Dot plot of normalized primary sphere (expressed as percentage) for combination treatment in BRAF-V600E MUT and WT lines. Secondary sphere assay was conducted with surviving cells from each treatment conditions from the primary sphere assay Panels (d-f). Panel (d) Quantification of the number of secondary spheres; Panel (e) the images of representative secondary sphere; Scale bar=100 μm. Panel (f). Dot plot of the relative number of secondary spheres in the combination wells for BRAF-V600E MUT and WT lines. In all melanoma lines, the combination treatment significantly reduced the primary and secondary sphere formation compared with all other treatments (DMSO or single drug). For visual clarity, we have not marked the significance in Panel (a) and Panel (d). * indicates p<0.05. Error bars represent +/−SEM.

FIG. 10 shows the combination-induced cell death was partially dependent on NOXA, BIM or BID. ATP assays with knockdown (KD) or knock out (KO) of indicated cells to test if the KD/KO protects against combination-induced cell death. Immunoblot to show the knockdown or knockout of NOXA, BIM or BID. Panel (a) KD lines for NOXA, BIM, or BID in A375. Panel (b) KD lines for NOXA in SKMEL-28. Panel (c) BIM KO lines in WM852c. * indicates p<0.05; ** indicates p<0.01. Error bars represent +/−SEM. Another pro-apoptotic BCL2 family member, BID, is the only member that can be activated by CASP8, which is one of the genes identified in the TCGA analyses. Therefore, we performed knock down experiments to investigate the role of BID in ABT-199 plus S63845 induced killing. Like NOXA and BIM, knockdown of BID also enhanced melanoma resistance to the combination (FIG. 5 Panel a). Taken together, our data indicated that BIM, NOXA and BID all play some roles in the killing induced by this combination.

FIG. 11 shows combination therapy of S64315/MIK665 (the clinic-ready version of S63845) with ABT-199 has a synergistic effect in treating melanoma samples of diverse genetic backgrounds and is comparable to the effects of S63845 with ABT-199. Panel (a), ATP assays of melanoma cell lines upon indicated treatments for 48 h. The viability of the DMSO control for each cell line was set to 100%. Both the combinations (S63845+ABT-199; upper panel and S64315+ABT-199, lower panel) had similar efficacy in reducing the cell viability of representative melanoma lines. Panel (b) Summary of ATP assay data of six melanoma cell lines, including patient derived cell lines, treated with single drug or a combination of S63845+ABT-199 or S64315+ABT-199. All drugs were used at a dose of 625 nM. For visual clarity, the * is not shown in the figures. Both combinations were highly synergistic at sub-micromolar doses. Error bars represent +/−SEM.

FIG. 12 shows uveal melanomas are very sensitive to MCL1 inhibitor alone, compared with cutaneous melanomas in vitro. Panel (a) ATP assays for uveal melanoma lines upon indicated treatments for 48 h. The viability of the DMSO control for each cell line was set to 100%. Panel (b) ATP assays for cutaneous melanoma lines upon indicated treatments for 48 h. The viability of the DMSO control for each cell line was set to 100%. Panel (c) Table for IC50 values of cell lines treated with MCL1 inhibitors.

FIG. 13 shows the treatment of MCL1 inhibitor S63845 significantly inhibited uveal melanoma tumor growth without affecting mouse weight. A uveal melanoma cell line, MP41, was used in the xenograft study, and tumor volume Panel (a) and weight of the mice Panel (b) were measured.

FIG. 14 shows lack of BAP1 expression in uveal melanoma did not alter sensitivity to MCL1 inhibitor. Panel (a) Immunoblot of BAP1 in various cell lines. Panel (b) Table for IC50 values of cell lines treated with MCL1 inhibitors. BAP1 null is associated with more aggressive forms of uveal melanomas.

FIG. 15 shows the treatment of MCL1 inhibitors plus BCL2 inhibitor ABT-199 significantly inhibited uveal melanoma tumor growth without affecting mouse weight. (panels a and b) ATP assays for various uveal melanomas upon 48 h indicated treatments of BCL2 inhibitor ABT-199 plus MCL1 inhibitor S63845 Panel (a) or MCL1 inhibitor S64315 Panel (b). The viability of the DMSO control for each cell line was set to 100%. Panel (c) A uveal melanoma line, MP41, was used in the xenograft study, and tumor volume and weight of the mice were measured. The combination's inhibiting effects on tumor volume was statistically significant, compared to vehicle or the single drugs (p<0.01).

FIG. 16 shows MCL1 inhibitors increased antitumor immunity and enhanced the efficacy of anti-PD1 ability to suppress tumor growth in the B16 in C57BL/6 syngeneic mouse model. Panel (a). MCL1 inhibitor S63845 inhibited tumor growth curve in vivo. 2.0×10⁵ B16F10 melanoma cells were injected in the flanks of 7-week-old C57BL/6J mice on day 0. MCL1 inhibitors was administered retro-orbitally daily at 40 mg/kg, starting at day 6 post tumor implantation. Panel (b). Flow cytometry of tumor-infiltrating immune cells of Veh (vehicle control) and 40 mg/kg MCL1 inhibitor S63845 treated tumors at day 10 post tumor implantation. Panel (c). MCL1 inhibitor S64845 inhibited tumor growth curve in vivo. Similar experiments as in a, except with i.p injection. Panel (d). Combination of MCL1 inhibitor S63845 and anti-PD1 suppressed tumor growth more than either drug alone. 2.0×10⁵ B16F10 melanoma cells were injected in the flanks of 7-week-old C57BL/6J mice. MCL1-i was administered through retro-orbital injection daily at 40 mg/kg on days 5-9, and anti-PD1 was injected intraperitoneally at 250 ug on days 7, 11, 14 and 16. Panels (e) and (f) show S64315 and anti PD-1 combination study. Panel (e) shows the combination treatment was significantly more effective than Vehicle and either drug alone. Panel (f) shows the combination treatment was well tolerated, as indicated by no dramatic weight loss for all the mice.

FIG. 17 shows that single-cell RNA-seq of tumor-infiltrated myeloid cells detected high MCL1 expression in the MDSC cell populations from two melanoma patients who relapsed from immunotherapies. Panel (a). UMAP clusters of tumor infiltrating myeloid cells analyzed by single cell RNA-seq. Red circle denotes the MDSC cluster identified as CD14+HLA-DR-CD33+IL-10+TGFB+ARG1+ cells. Panel (b). UMAP defined clusters colored based on the expression of MCL1. Red circle denotes the MDSC cluster.

FIG. 18 shows T cell proliferation is maintained in the presence of physiologic concentrations of MCL1 inhibitor. Quantification of the percentage of dividing T cells from human PBMCs stimulated (Stim) with anti-CD3/CD28 microbeads in the presence of IL-2 and increasing does (0.156-10 uM) of S64315 (S64) or DSMO vehicle control. At physiologically relevant doses, S64 did not alter T cell proliferation.

FIG. 19 is a table showing comparison of the mRNA expression from the TCGA cutaneous melanoma data set. Multiple t-test for false discovery rates (FDR). Two-stage linear step-up procedure of Benjamini, Krieger and Yekutieli, with Q=2.5%. Each row was analyzed individually, without assuming a consistent SD.

FIG. 20 is a table showing comparison of the protein expression from the TCGA cutaneous melanoma RPPA data set. Multiple t-test for false discovery rates (FDR). Two-stage linear step-up procedure of Benjamini, Krieger and Yekutieli, with Q=2.5%. Each row was analyzed individually, without assuming a consistent SD.

### DETAILED DESCRIPTION

This disclosure is related to methods of using BH3 mimetics in combination with immunotherapies to enhance immune function of a patient in need thereof. In many embodiments, the disclosed therapeutic compounds and treatments may be useful in decreasing the abundance or activity of immunosuppressive cells and increasing the abundance or activity of immunostimulatory cells. In several embodiments, a BH3 mimetic may help to increase the number of tumor-infiltrating immune cells and/or the immunogenicity of antigen presenting cells. In many embodiments, the disclosed compounds, compositions, and methods may help in improving the efficacy of immunotherapies, especially in the case of patients that have failed to respond to, or relapsed after standard treatments. In this manner, the combination of a BH3 mimetic with an immunotherapy enhances immune function.

The disclosed compositions and methods may be useful in treating various diseases, disorders, and indications. In some embodiments, the disclosed compositions and methods may be useful in enhancing immune function to combat the disease, disorder, or indication. In some embodiments, the disease, disorder, or indication is selected from a cancer, such as melanoma, cervical, lung cancer, kidney cancer, Hodgkin lymphoma, head and neck squamous cell cancer, advanced bladder cancer, advanced liver cancer, advanced esophageal squamous cell cancer, colorectal cancer, small cell lung cancer, non-small cell lung cancer, cutaneous squamous cell carcinoma, breast cancer, Merkel cell carcinoma, advanced lung cancer, advanced kidney cancer, and head and neck cancer. In some embodiments the diseases, disorders, and indications may be virally induced.

### Therapeutic Compounds

Disclosed herein are compositions containing, and methods of using, therapeutic compounds to enhance immune function. Therapeutics of the present disclosure target pathways that may suppress or inhibit proper immune function (e.g., suppress the immune system and/or immune cells). As one example, therapeutic compounds of the present disclosure target and suppress pathways and cells that may inhibit T cell responses, for example T cell recognition of tumor cells. The disclosed therapeutic compounds may aid in reducing or stopping the immunosuppression of T cells to promote an immune system attack on tumor cells.

In many embodiments, the disclosed therapeutic compounds may be BH3 mimetics. In many embodiments, the disclosed BH3 mimetics may help regulate a patient's immune response. BH3 mimetics are small-molecule drugs that mimic the function of the pro-apoptotic BH3 group (Group 3). These mimetics inhibit the activity of the pro-survival BCL-2 family members (Group 2), thus initiating apoptosis. Currently, several BH3 mimetics, including those inhibiting BCL2 (ABT-199/venetoclax), MCL1 (S63845/S64315, ABBV-467, AZD-5991, AMG-176, AMG-397), or BCL2/BCLXL/BCLW (ABT-263), are being tested or are clinically available as cancer treatments. BH3 mimetics offer alternative options for patients who may not respond to current cancer treatments, as the BH3 mimetics act through different mechanisms. As one example, in therapy resistant acute myeloid leukemia patients, BH3 mimetics have increased the response rate to as high as 91%. As another example, GX15-070 (a pan inhibitor of MCL-1/BCL-2/BCL-XL/BCL-W/BFL-1) preferentially induces cell death of regulatory T cells (Tregs) and enhances clearance of lung cancer cells when combined with a tumor vaccine. In some embodiments, the therapeutic compound may be selected for affinity to a BCL2 family member. In various embodiments, the therapeutic compound is one or more of S64315, S63845, AMG-176, and AZD5991. In many embodiments, the therapeutic compound is S64315 or S63845.

MCL1 inhibiting compound, MCL1 inhibiting BH3 mimetic, and other similar terms may refer to various compounds that inhibit the function of the MCL1 protein. In some embodiments, the disclosed compounds inhibiting MCL1 may include S63845, S64315, ABBV-467, AZD-5991, AMG-176, AMG-397.

BCL2 inhibiting compound, BCL2 inhibiting BH3 mimetic, and other similar terms may refer to various compounds that inhibit the function of the BCL2 protein. In some embodiments, the disclosed compounds inhibiting BCL2 may include ABT-199/venetoclax, and ABT-263/navitoclax.

Studies tend to focus on the ability of BH3 mimetics to directly kill tumor cells; however, treatments with single BH3 mimetics are not effective at eliminating solid tumors. Disclosed herein is the combination of the presently disclosed therapeutic compounds (for example BH3 mimetics) with a second therapeutic composition to enhance elimination of solid tumors. For example, a therapeutic of the present disclosure combines a BH3 mimetic with an immunotherapy to improve the efficacy of the immunotherapy in targeting the tumor cells. In several embodiments, myeloid-derived suppressor cells (MDSCs) may be targeted by the present therapeutic compounds (including, for example, one or more BH3 mimetics) to improve immune response, increase clinical response to immunotherapy, and improve overall survival in cancer patients, such as, for example, melanoma patients.

In several embodiments, the disclosed therapeutic compounds may help to reduce MDSC frequency while increasing CD8+ T cell number and antitumor activity. In many embodiments, use of the disclosed therapeutic compounds, methods, and treatments may decrease the abundance or activity of immunosuppressive cells, reducing or eliminating suppression of a patient's immune system, immune function, and/or T-cells, for example by decreasing the number, abundance, or concentration of inhibitory MDSC cells. In many embodiments the number, concentration, or abundance of MDSC cells may be reduced by about 10-99%, for example greater than about 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99%, and less than about 100%, 99%, 98%, 97%, 96%, 95%, 94%, 93%, 92%, 91%, 90%, 85%, 80%, 75%, 70%, 65%, 60%, 55%, 50%, 45%, 40%, 35%, 30%, 25%, 20%, or 15%.

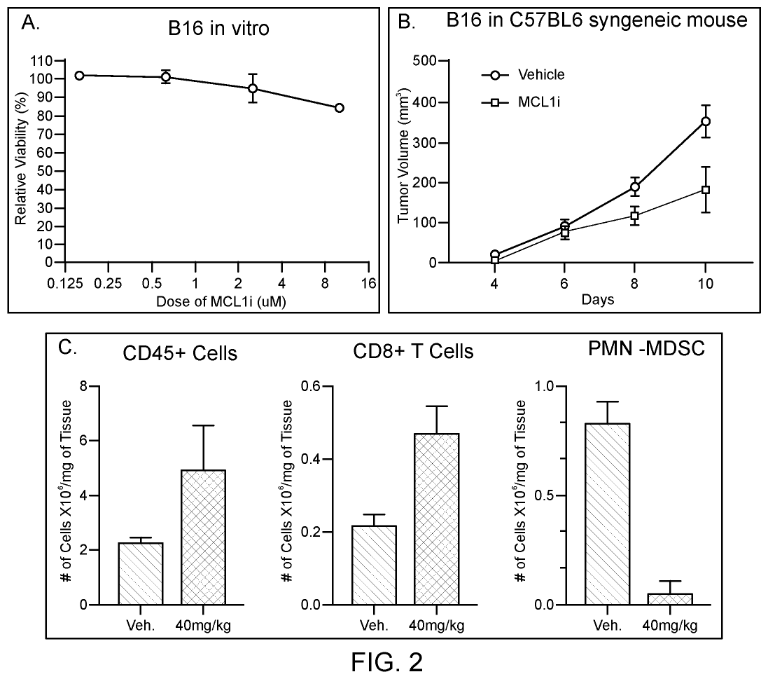

The disclosed therapeutic compounds may help enhance immunotherapy treatment to increase immune activation within the tumor and/or tumor clearance. In several embodiments, an MCL1 inhibitor (MCL1i) is used in a therapeutic to improve antitumor immunity and enhance efficacy of immunotherapies. MCL1 is a BCL2 family protein that promotes survival and inhibits apoptosis. For example, MCL1 is important for the survival and function of MDSCs and regulatory T cells (Tregs). As discussed, MDSCs are a significant source of immunosuppression in tumors. CD8+ T cells, when not immunosuppressed (e.g., by MDSCs), are tumor-eliminating immune cells. In some embodiments, wherein the disclosed compositions and methods enhance immune function or improve immune system response by increasing the number, concentration, or abundance of one or more immune cell types, for example without limitation CD45+ and CD8+ T-cells. In many embodiments, the number, concentration, or abundance of these cells may increase, in response to treatment with the disclosed compositions and methods, by about 10-200% for example greater than about 10%, 20%, 30%, 40%, 50%, 60%, 70%, 80%, 90%, 100%, 110%, 120%, 130%, 140%, 150%, 160%, 170%, 180%, 190%, 200%, and less than about 200%, 190%, 180%, 170%, 160%, 150%, 140%, 130%, 120%, 110%, 100%, 90%, 80%, 70%, 60%, 50%, 40%, 30%, 20%, 10%, after about 1-4 weeks, for example more than 6, 8, 10, 12, 14, 16, 18, 20, 22, 24, 26, 28, or 30 days, and less than about 32, 28, 26, 24, 22, 20, 18, 16, 14, 12, 10, or 8 days. In one embodiment, as shown in FIG. 2, Panel C, the number of CD8+ T-cells may increase about 100%-150%.

Certain viruses can induce MCL1 expression in infected cells or induced cancers to promote cell survival. MCL1 inhibitors can be used to treat these infection and cancers induced by these viruses.

MCL1i is a BH3 mimetic that may inhibit MCL-1 to alter the population of tumor-infiltrating immune cells and/or the immunogenicity of tumor cells. For example, MCL1i may be administered to a patient to drastically decrease the frequency of MDSCs and increasing the frequency of tumor-infiltrating CD8+ T cells (FIG. 2C). With reduced numbers of MDSCs suppressing the tumor-eliminating immune cells and an increased frequency of tumor-eliminating immune cells, the tumor-eliminating immune cells can partially or completely block tumor growth in vivo.

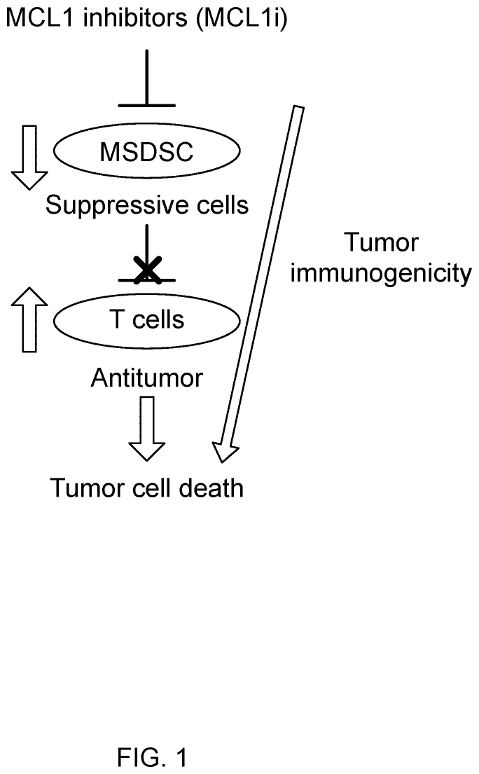

Multiple mechanisms can lead to decreases in MDSCs and increases in CD8+ T cells upon MCL1i treatment. For example, as shown in FIG. 1, 1) MCL1i may kill MDSCs but not CD8+ T cells (e.g., removing suppression of T cells and allowing T cells to induce tumor cell death); 2) MCL1i may inhibit MDSC suppressive function while increasing CD8+ T cell activity; and/or 3) MCL1i may increase tumor cell immunogenicity, making the tumor cells more visible to the immune system.

The disclosed therapeutic compositions may be administered in various ways and at various dosages. In some embodiments, the disclosed therapeutic compounds may be administered at about 1 to 100 mg/kg, for example about 40 mg/kg. In some embodiments, the dosing may be greater than about 10 mg/kg, 15 mg/kg, 20 mg/kg, 25 mg/kg, 30 mg/kg, 35 mg/kg, 40 mg/kg, 45 mg/kg, 50 mg/kg, 55 mg/kg, or 60 mg/kg, and less than about 65 mg/kg, 60 mg/kg, 55 mg/kg, 50 mg/kg, 45 mg/kg, 40 mg/kg, 35 mg/kg, 30 mg/kg, 25 mg/kg, 20 mg/kg, or 15 mg/kg. In many embodiments, the disclosed therapeutic compounds may be administered orally or by injection, for example intravenous, intra-arterial, retro-orbital, subcutaneous, or intraperitoneal or any combination thereof.

### Methods of Treatment

Therapeutic compounds and methods described herein may be used to improve treatment outcomes for diseases and disorders with an immunological component, such as many cancers. For example, therapeutics of the present disclosure may be used to enhance treatment of various solid tumor cancers, such as melanoma. As one example, a therapeutic of the present disclosure may enhance the patient's immune response to solid tumors. An enhanced response may be determined by comparing the size of solid tumors (for example by volume) for a population of patients not treated with the presently disclosed compounds, compositions, and/or methods, to a similar population of patients that have been treated with the presently disclosed compounds, compositions, and/or methods—in these cases, the population of patients treated with the disclosed compounds, compositions, and/or methods have an average tumor volume that, over the same treatment period, is less than about 75%, 70%, 65%, 60%, 55%, 50%, 45%, 40%, 35%, 30%, 25%, 20%, 15%, 10%, or 5% that of the untreated population. In some embodiments this may be referred to as rejecting, eliminating, or reducing the solid tumor. In some cases elimination may be referred to a radiologically undetectable tumor. In these cases, the period of treatment may be about 30 to 90 days, for example greater than about 25 d, 30 d, 35 d, 40 d, 45 d, 50 d, 55 d, 60 d, 65 d, 70 d, 75 d, 80 d, or 85 d, and less than about 90 d, 85 d, 80 d, 75 d, 70 d, 65 d, 60 d, 55 d, 50 d, 45 d, 40 d, 35 d, or 30 d. In these embodiments, the disclosed compounds and methods may help to reduce or eliminate suppression of a patient's immune cells. In many embodiments, use of the disclosed compounds, compositions, and methods to treat a patient may have little or no effect on the patient's weight, for example the average weight of a population of treated patients may be within about 1-20%, for example 1-10%, or 1-5% of the average weight of a population of patients receiving no treatment, or only one part of the disclosed treatment, for example greater than about 1%, 2%, 3%, 4%, 5%, 6%, 7%, 8%, 9%, 10%, 11%, 12%, 13%, 14%, 15%, 16%, 17%, 18% or 19% and less than about 20%, 19%, 18%, 17%, 16%, 15%, 14%, 13%, 12%, 11%, 10%, 9%, 8%, 7%, 6%, 5%, 4%, 3%, 2%, 1%.

The disclosed therapeutic compounds and methods may be combined with other therapies to enhance the effectiveness of those therapies. In most embodiments, these combination therapies may include BH3 mimetics, immunotherapies, etc. In one embodiment, the disclosed compounds and methods may aid in therapies involving treating a patient with a therapeutic compound, such as an immunotherapeutic compound, for example an antibody, that is directed to a cancer cell.

Therapeutics described herein may be used to enhance vaccination treatment and efficacy. For example, vaccines may be used to treat viral or, in some cases, bacterial infections caused by the introduction of pathogens in the body. A vaccine may induce an immune system response to such pathogens by promoting the production of antibodies and/or T-cells. The therapeutics and methods of the present disclosure may enhance a patient's response to vaccination treatment by reducing or eliminating suppression of a patient's immune system.

### Antitumor Immunity

A subject's immune response may be blunted by one or more immunosuppression mechanism. One mechanism is through the recruitment and expansion of myeloid-derived suppressor cells (MDSCs). MDSCs comprise a heterogeneous population of immature immunosuppressive myeloid lineage cells that are typically increased in tumor-bearing hosts and can be a significant source of immunosuppression. For example, MDSCs have been identified as an obstacle to the successful immunotherapeutic treatment of advanced melanoma. That is, MDSCs may act to suppress the body's attempt to attack melanoma cells. MDSCs utilize a wide repertoire of suppressive mechanisms to prevent antitumor immunity. In one example, MDSCs may suppress or inhibit tumor-eliminating immune cells, such as CD8+ T-cells.

Tumor cells may evade an immune system attack in various ways. For example, tumor cells may express proteins typically expressed on normal cells. One such class of proteins are proteins that may interact with T-cells investigating the cell, such as immune checkpoint proteins. When T cells investigate tumor cells expressing checkpoint proteins, the T cell may not react to the tumor cell, allowing the tumor cells to avoid attack.

### Checkpoint Inhibitors

Checkpoint inhibitors typically bind checkpoint proteins and block their recognition by T cells. This allows the T cells to recognize the tumor cells as abnormal and mount an attack on the tumor cells.

PD-1 is a checkpoint protein on T cells that binds another checkpoint protein, PD-L1, on most normal cells. When PD-1 binds to PD-L1, PD-1 acts as an “off switch” to keep T cells from attacking cell expressing PD-L1. As described above, some cancer cells evade the immune system by expressing PD-L1 on their surface. PD-1 inhibitors or PD-L1 inhibitors respectively target PD-1 and PD-L1 to block this interaction. In many cases, PD-1 and PD-L1 inhibitors may be monoclonal antibodies or immunoglobulins with affinity for the surface proteins and able to disrupt or prevent binding between PD-1 and PD-L1.

### BCL2

BCL2 genes are important for the function and survival of various immune cells. The BCL-2 family codes for three groups of proteins. The groups are differentiated based on their structure and function: Group 1—contain multiple BH domain containing proteins that promote apoptosis; Group 2—promote survival and inhibit apoptosis (MCL1, BCL2, BCLXL, BCLW, and BFL1); and Group 3—contain only the BH3 domain, and promote apoptosis. Together, interactions between members of these different groups control the initiation of apoptosis (i.e., programmed cell death).

### MCL1 Inhibiting Compounds

Disclosed herein is the use of MCL1 inhibiting compounds to kill or inhibit growth of various melanoma cells. In some embodiments, the disclosed compounds may be used to inhibit growth of or kill uveal melanoma in vitro and in vivo. While, in some cases, loss of BAP1 may be associated with more aggressive uveal melanoma, loss of BAP1 in the presently treated cells did not alter sensitivity to the disclosed MCL1 inhibitor compounds. These results suggest that MCL1 inhibitors may be effective with even very aggressive melanomas, including uveal melanomas. Some mucosal melanomas may also be treated with the disclosed MCL1 inhibitors. In one case, a mucosal melanoma, MB3443, was shown to be sensitive to the disclosed MCL1 inhibitors with IC 50 of 0.05 uM. Currently, there are no FDA approved drugs for treatment of uveal melanoma.

### Combination of BCL2 and MCL1 Inhibitory Compounds

Combinations of MCL1 inhibitor compounds and BCL2 inhibitor compounds (that is compounds with affinity for, or that bind to MCL1 or BCL2) are shown herein to be effective therapeutic options for patients with advanced melanoma. In many embodiments, melanomas lacking mutations in BRAF, such as hotspot BRAF-V600 mutations, may be more sensitive to the disclosed therapy. In some cases, treatment of cells with BCL2 inhibitory compounds enhanced cell sensitivity to a MCL1 inhibitor, and treatment with MCL1 inhibitory compounds sensitized cells to a BCL2 inhibitor. This data indicates that targeting both MCL1 and BCL2 simultaneously was useful in kill melanoma cells. In vitro, the disclosed combination is efficient in killing (inducing death) of melanoma cells. In many embodiments, melanoma cells lacking BRAF-V600E/K mutations demonstrated heightened sensitivity to the disclosed combination therapy. In vivo, the disclosed combination therapies inhibited tumor growth synergistically when compared to either individual compound.

### Target Tissue and Cells

The disclosed therapies may be useful in treating various malignant cells and tissues. In some embodiments, the targeted cells and tissues may be a tumor. In some embodiments, the cells and tissues may be selected from one or more of melanoma, small cell and non-small cell lung cancer, Hodgkin lymphoma, head and neck squamous cell carcinoma, cutaneous squamous cell carcinoma, locally advanced cutaneous cell carcinoma, urothelial carcinoma, merkel-cell carcinoma, lung cancer, kidney cancer, advanced bladder cancer, advanced liver cancer, advanced esophageal squamous cell cancer, colorectal cancer, breast cancer, advanced lung cancer, advanced kidney cancer, and tumors with high non-synonymous mutation burden. In many embodiments, the cells and tissues are melanoma related, including cutaneous melanoma and subtypes of uveal, acral, and mucosal melanomas. In many embodiments, the disclosed therapies may be useful in killing, inducing death, and/or slowing the growth of primary and metastatic melanoma cells.

The disclosed compounds and therapies may be effective at killing, inducing death, and/or inhibiting growth of various cells, for example tumor cells and cells that may possess stem-like characteristics. In some embodiments the disclosed compounds may be used to treat Melanoma Initiating Cells (MICs), which may contribute to drug resistance and relapse. In some embodiments these cells may be cells with sphere-forming capacity, and a sub-population of melanoma cells having enhanced plasticity, drug resistance and stem-cell-like features. Thus, the disclosed compounds and therapies are also effective at reducing relapse.

The disclosed compounds and therapies may be effective at targeting myeloid-derived suppressor cells (MDSCs). In some embodiments, the disclosed compounds and therapies may suppress the growth of, induce death, and/or kill MDSCs, which may be a heterogeneous population of immature and/or immunosuppressive myeloid cells. MDSCs may be a significant source of immunosuppression in tumors.

### Biomarkers

The disclosed cells and tissues targeted for treatment with the disclosed therapies may have various genetic and/or biomarkers. In many embodiments, these genetic and biomarkers may indicate that the cells, tissues, and tumors possess a heightened sensitivity to the disclosed therapies. In some embodiments, melanoma cells lacking BRAF mutations, for example, BRAF hotspot mutations, and/or BRAF-V600E/K mutations may be treated with the disclosed therapies. In some embodiments, cells expressing high levels of MCL1 may be especially sensitive to the disclosed therapies, especially MCL1 inhibitor compounds. In some embodiments, MDSC cells expressing high levels of MCL1 in the tumor microenvironment may be treated with the disclosed compounds, for example MCL1 inhibitor compounds to enhance the ability of the host immune system to kill malignant cells.

Treatment options for patients with advanced cutaneous melanoma expanded significantly with FDA approval of immune-checkpoint blockade drugs and BRAF targeting signal transduction inhibitors. Despite these advances, a substantial fraction of patients is not eligible for these treatments. This includes those with BRAF-WT melanoma, and those who do not respond to immunotherapies. In addition, patients with rare melanoma subtypes, such as acral and mucosal, are genetically distinct from cutaneous melanomas and typically lack BRAF-V600E/K mutations. A large proportion of these patients also do not respond to immunotherapies. Therefore, there is a significant need to develop new treatments for these patients. Our study strongly suggests that combinations of BH3 mimetics that target both MCL1 and BCL2 fill this role and in some cases detection of BRAF-WT maybe be used to select patients.

Analyses of TCGA data identified higher expression of BCL2 in cutaneous BRAF-WT melanoma, compared to those with a BRAF hotspot mutation. These data led to exploring BH3 mimetics in advanced melanomas, especially those without BRAF-V600E/K (i.e. BRAF hotspot mutations). For this reason, analysis was expanded to include rare melanoma subtypes of mucosal and acral, which often lack BRAF hotspot mutations. In vitro and in vivo data showed that the simultaneous targeting of BCL2 and MCL1 is effective in treating advanced melanoma, and this combination is more potent in melanomas without the BRAF-V600E/K variant. This observation is novel and provides a feasible approach for patients with limited therapeutic options.

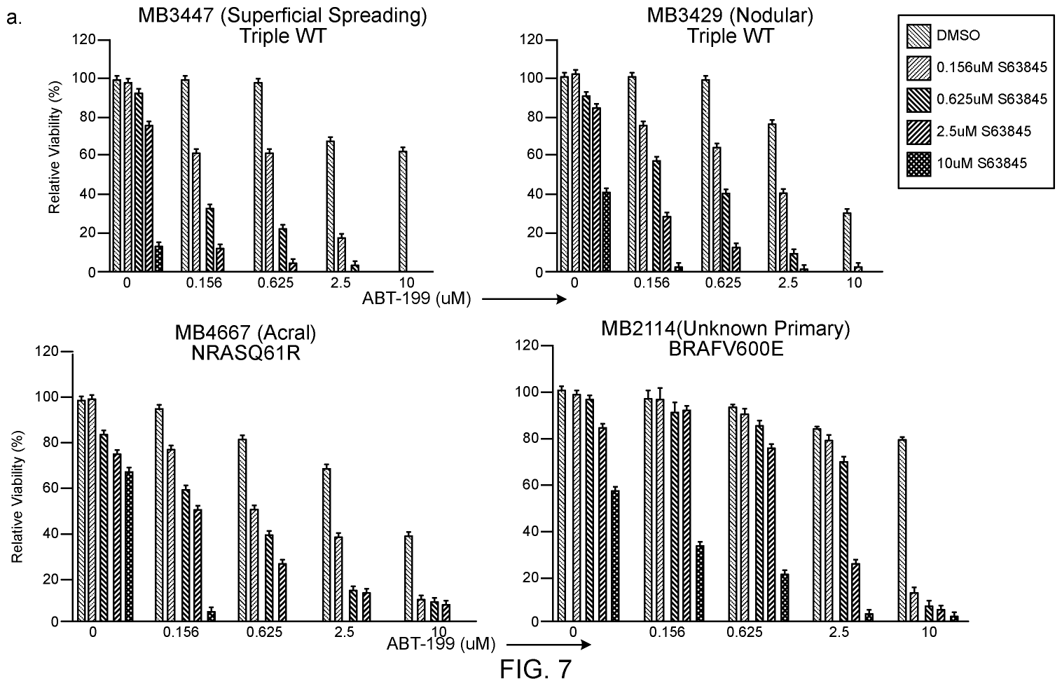

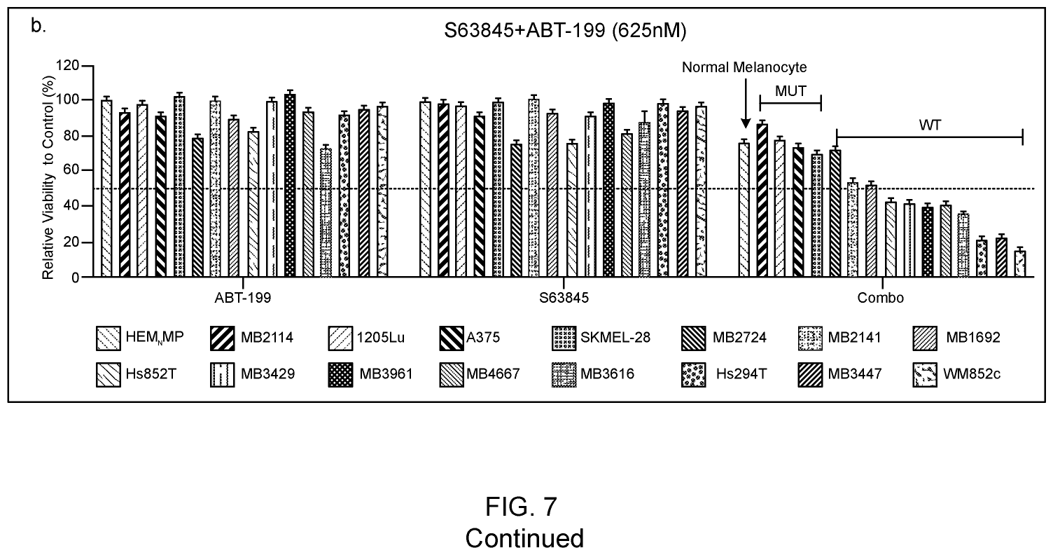

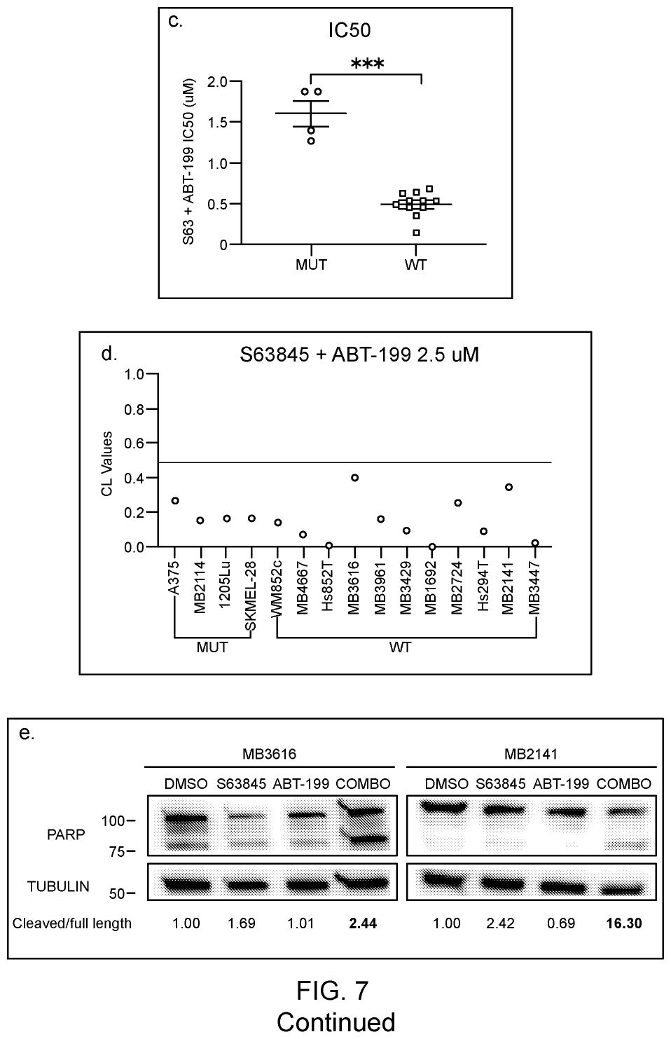

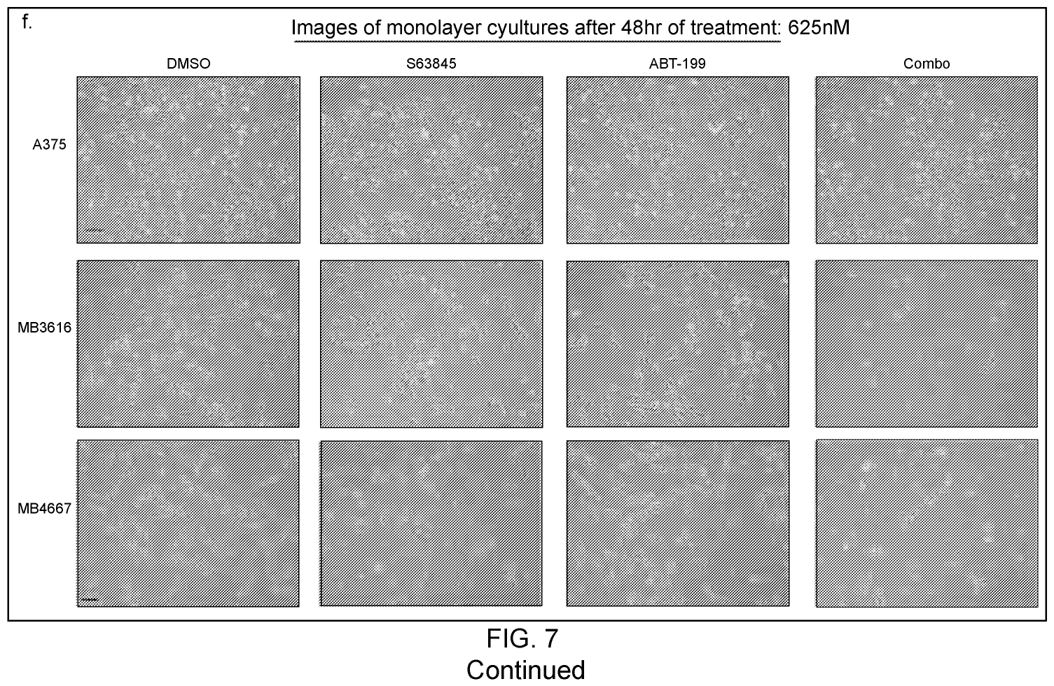

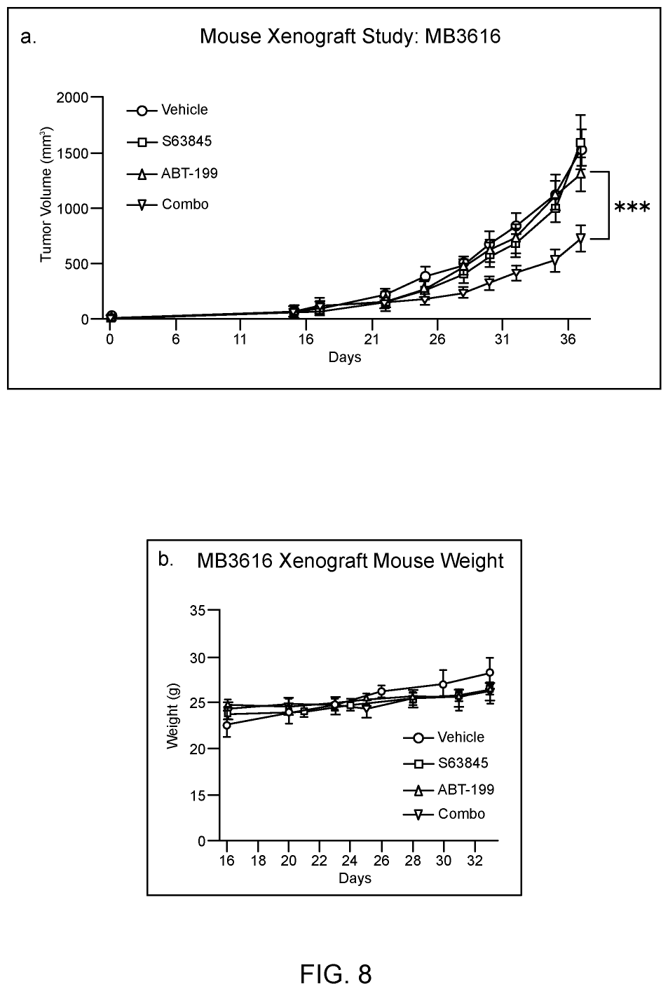

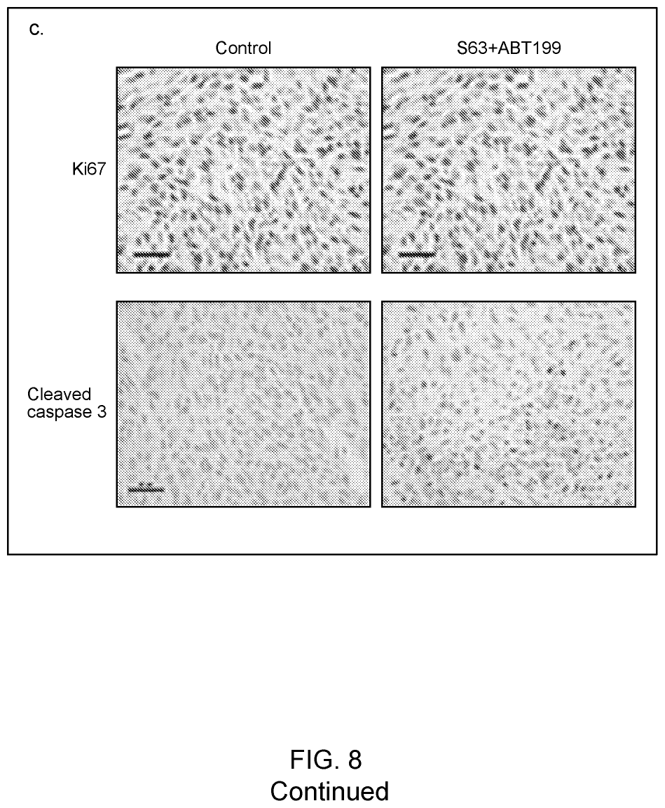

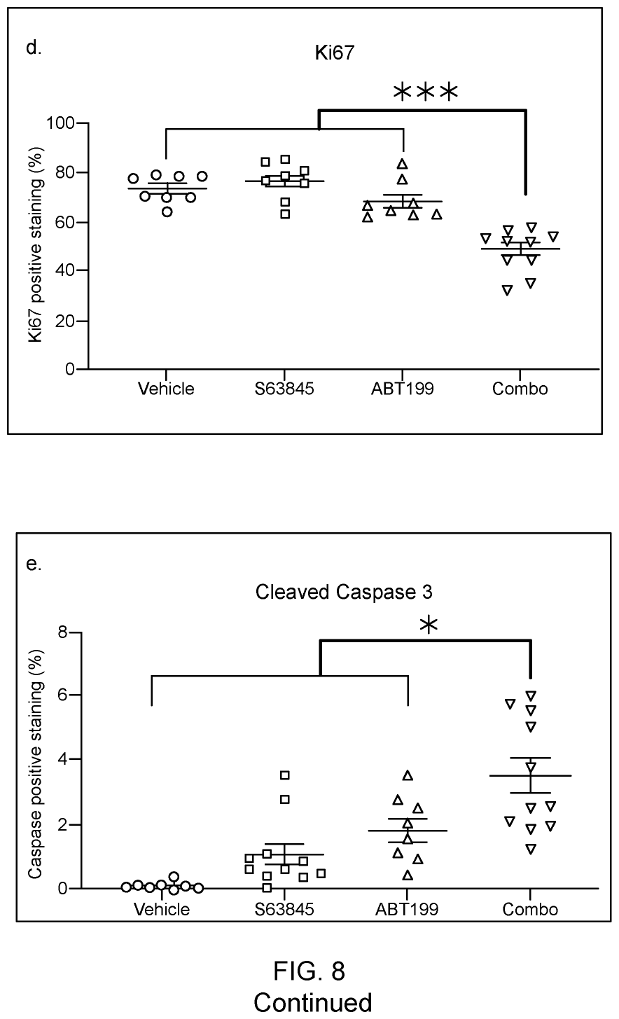

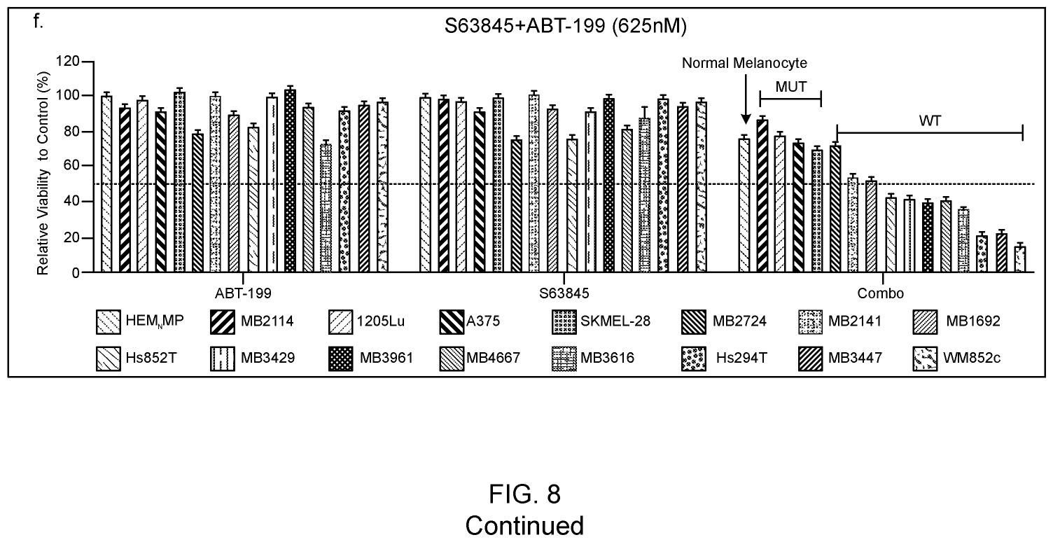

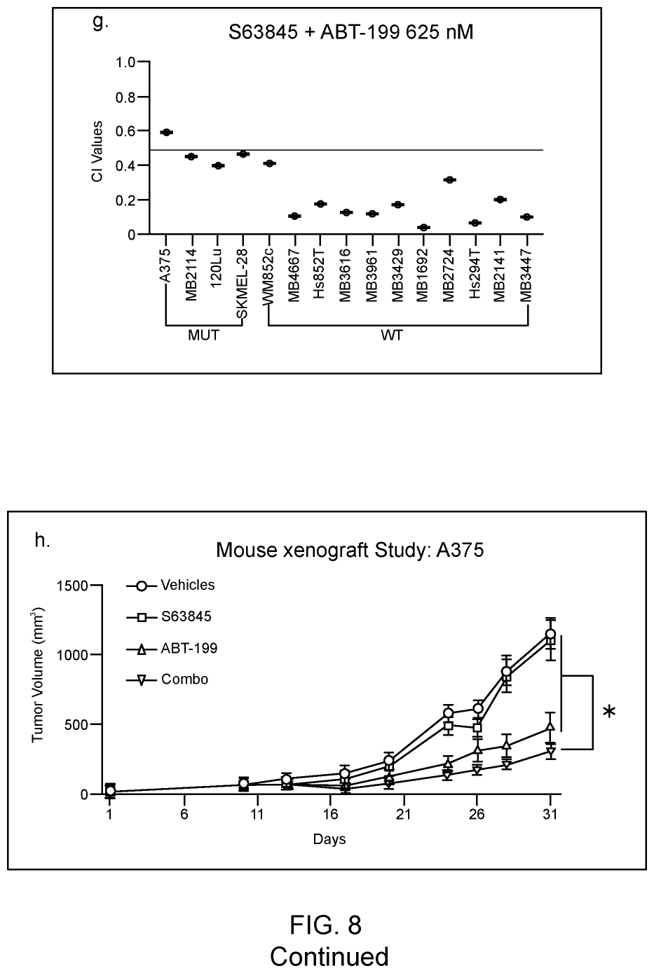

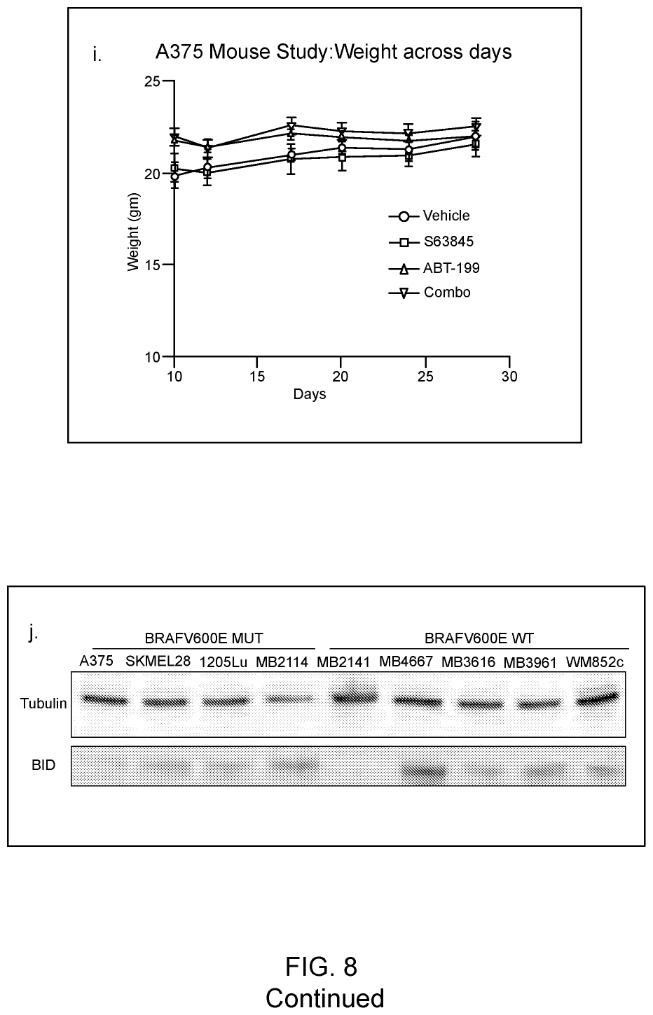

This combination was successful across the panel of melanoma cell lines. However, the combination of an MCL1 inhibitor and ABT-199 was more effective in melanomas with the BRAF-WT genotype. For BRAF-V600E melanoma models, while effective, higher doses and/or increased frequency of administration was needed for the disclosed combination to achieve similar results—i.e. a reduction in cell viability in vitro and/or inhibition of tumor growth in vivo (FIG. 7 and FIG. 8 panels a-i). There was minimal toxicity in vivo. Even with more frequent drug administration, any obvious adverse health effects such as weight loss were not detected in mouse xenograft model. These data indicate that the drugs could be delivered more frequently and/or at a higher concentration to achieve similar results in patients with BRAF mutated melanoma.

This is the first study of the efficacy of the combination of S63845 with ABT-199 in vivo in melanoma. In addition, data with sphere assays suggest this combination may prevent relapse caused by MICs. Moreover, the study also includes in vitro data that tests a clinic-ready version of the MCL1 inhibitor, S64315 (MIK665), in combination with the FDA approved ABT-199. Further justifying the use of this therapeutic option for patients with advanced melanoma. It was shown that navitoclax (a BCL2/BCLW/BCLXL inhibitor; also ABT-263) plus MCL1 inhibitors can be effective to kill melanomas in vitro and in vivo, however, the potential toxicity was higher than ABT-199 plus MCL1 inhibitors. In the earlier study, the drug concentrations of navitoclax and MCL1 inhibitors were lowered to minimize toxicity. In comparison, the combination of ABT-199 plus an MCL1 inhibitor has been tested in pre-clinical mouse studies and is considered safe even when administered at higher concentrations than what is demonstrated in this current study (S63845: 25 mg/kg twice weekly; ABT199: 50 mg/kg 2-3 times per week). For instance, Seiller et al. used ABT-199 (100 mg/kg; 5 days/week) along with S63845 (25 mg/kg; every 6 days) for 19 days without significant weight loss in NSG mice. Prukova et al. safely administered S63845 (25 mg/kg) and ABT-199 (50 mg/kg) simultaneously in a mouse model of AML for five days. Minimal toxicity was observed with other clinically approved MCL1 inhibitors (such as AZD5991), which were administered at higher doses and more frequently than in the current study. In addition, ABT-199 is already FDA-approved for some cancers, and a phase 1 study (NCT03672695) examining the combination of S64315 with ABT-199 is underway for patients with AML. The study protocol includes the weekly administration of S64315 (50 mg-1000 mg) and daily ABT-199 (100-600 mg). Notably, no toxicity has been reported to date, supporting what the in vivo models show.

BH3 mimetics promote cell death primarily through the intrinsic apoptotic pathway. Our data indicate that BIM, NOXA, and BID play a role in this combination treatment. BIM and NOXA's roles are consistent with the primary apoptotic pathway promoted by BH3 mimetics. Although knockdown of BID decreased cell sensitivity to this treatment, data indicates that a BID-null state will not prevent killing completely, as BID was not detected in the moderately sensitive cell line MB 2141 (FIG. 8 panel j). The role of BID has only been reported in the effects induced by the BH3 mimetic ABT-737. BID provides a link between the extrinsic and intrinsic apoptotic pathways. These data indicate that crosstalk between these two apoptosis pathways is involved with BH3 mimetic treatments.

To explore why BRAF-WT melanomas respond better to this combination, the expression of various BCL2 family members were examined. There was a higher expression of BIM in BRAF-WT melanoma, compared to BRAF-MUT melanoma, although it is not statistically significant (Data not shown). This is consistent with previous finding that BRAF-V600E can downregulate BIM expression in melanoma. Considering that the data showing BIM knockout or knockdown results in decreased melanoma sensitivity to this combination, higher BIM expression may be a contributing factor for a better response in BRAF-WT melanoma and may therefore be useful to identify patients who will respond well to this treatment.

### EXAMPLES

### Materials and Methods

Analysis of the TCGA cutaneous melanoma dataset: Data and clinical information were downloaded from the FireBrowse website (firebrowse.org/) and supplemental files provided in the TCGA cutaneous melanoma paper. Multiple t-tests were performed using GraphPad v8, discovery determined using the Two-stage linear step-up procedure of Benjamini, Krieger and Yekutieli, with q=1%. Each gene or protein was analyzed individually, without assuming a consistent standard deviation.

Reagents and drug treatments: All drugs (S63845, S64315, and ABT-199) used for the study were purchased from MedChem Express (Monmouth Junction, N.J.) or from Selleck Chem (Houston, Tex.). For the initial cell viability assays, each drug was tested at a dose range of 0.156 to 10 μM by itself or in combination, and then a dose of either 0.625 or 2.5 μM was used for the subsequent studies. Cells were treated for 48 h for viability assays and primary sphere assays.

Melanoma cell lines, either long-established conventional lines or newly established patient lines: Patient derived cell lines were provided by the University of Colorado Skin Cancer Biorepository (patient consent and specimen usage outlined under COMIRB 05-0309) and validated by melanoma triple cocktail staining. Patient lines were derived from metastases of patients seen at our institution, and include samples derived from patients relapsed from current treatments. Patient lines were STR profiled with >80% match to the patient's corresponding blood sample. Genetic backgrounds are listed in Table 1, below. All cell lines were maintained in RPMI 1640 medium (Invitrogen, Grand Island, N.Y.) with 10% fetal bovine serum (Gemini Bio-Products, Inc., West Sacramento, Calif.) and were tested for mycoplasma. Primary melanocytes HEMNMP were obtained from Life Technologies (Carlsbad, Calif.). Melanocytes were maintained in Medium 254 with Human Melanocyte Growth Supplement-2 (Life Technologies, Carlsbad, Calif.). To mimic melanoma culture conditions, 10% FBS was added for drug assays.

ATP viability assay, primary and secondary sphere assays: Cell viability was evaluated via Cell Titer-Glo Luminescent cell viability assay (Promega Corp., Madison, Wis.) according to the manufacturer's protocol. All sphere assays were completed as previously described. The experimental schematic for the primary and secondary sphere assays was described previously. All assays were performed in no less than triplicate for each cell line and repeated at least three times for each line. Drug treatments began 120 h after seeding in the primary sphere assay and 24 h after seeding for the monolayer ATP assay.

Immunoblot: Both floating and adherent cells were collected and lysed using 2× Laemmli buffer (Bio-Rad, Hercules, Calif.). Samples were analyzed using the immunoblot analysis protocol as described earlier. Immunoblot images presented in this manuscript are from a representative experiment carried out in triplicate. The antibody dilutions were used at the manufactures' recommendation. The following antibodies were purchased from Cell Signaling Technologies (Danvers, Mass.): PARP 340 (#9532), BID (#2002), BIM (#2933), BCL2 (#15071), α/β TUBULIN (#2148), and HRP-conjugated goat anti-mouse and anti-rabbit antibodies. The NOXA antibody (#OP180) was obtained from Millipore Sigma (St. Louis, Mo.) and MCL1 antibody (#819) was purchased from Santa Cruz Biotechnology (Dallas, Tex.).

Creation of short hairpin RNA transduced cell lines and CRISPR/Cas9-mediated BIM knockout cell lines: ShRNA lentiviral particles (Santa Cruz Biotechnology, Dallas, Tex.) were used to construct stable cell lines as previously described. BIM-knockout lines were generated using CRISPR/Cas9 technology, as previously described.

Mouse xenograft studies: All animal experiments mentioned in this study were approved by the Institutional Animal Care and Use Committee (IACUC) of the University of Colorado Denver (protocol number 318). Similar mice and standard methodology were used for tumor implantation and tumor measurements, as above. Briefly, 6-8 week old NCRNU nude mice were used, with tumor cells injected subcutaneously in each flank with a 100 ul suspension of 2 to 3.5 million cells in 50% BD Matrigel (#354263, BD Biosciences). Drug treatments started when the tumors were palpable. Treatment groups consisted of randomly divided mice of at least 8 tumors each group. All drugs were prepared according to the manufacturer's protocol, or previously described. S63845 was administered at 25 mg/kg twice weekly. ABT-199 was administrated 50 mg/kg twice per week for the study with MB3616 cells and three times per week for the study of A375 cells. The tumor samples were collected at the end of the experiment for further studies.

Immunohistochemistry (IHC): The detailed procedure and the protocol have been previously described. In short, the tumors were subjected to fixation and dehydration gradient before being embedded with paraffin. 4-μm thick sections were used for staining in a Dako Autostainer as previously described. The antibodies used in the study are Cleaved Caspase-3 (1:200, #9664 Cell Signaling Technology) and Ki67 (1:100 #RM-9106-S1, Thermo Fisher Scientific). The details of imaging and quantifications are also previously described.

Calculation of IC50 and Combination index (CI) values: IC50 values were calculated from the relative viability results of ATP assays, and were derived by a sigmoidal dose (log)-response (variable slope) curve with GraphPad Prism 8 software. The combination index was calculated to determine the synergistic effects of the combination treatment, using Compusyn software (version 1; available at www.combosyn.com); values <0.9 indicate synergism and smaller CI values indicate stronger synergy (Chou, T C 2006). The combination index (CI) is described in Chou T. C. and Talalay P., “Quantitative analysis of dose-effect relationships: the combined effects of multiple drugs or enzyme inhibitors,” Advances in Enzyme Regulation, Vol. 22, pp. 27-55, 1984, Pages 27-55. This method uses a median-effect equation and Combination-index theorem to calculate synergy between two drugs and identifies the drug interaction as values ranging from 0 to 1. This is defined as combination-index values (CI values). The program requires entering a series of “dose (D) and effect (fa)” into the program for each drug alone as well as the combination therapy. From this data, the software calculates the CI values at different fa levels based on the CI algorithm. In many embodiments, the disclosed compositions, compounds, and methods result in synergy (more than additive results) over administration of individual members of the compositions, in some embodiments synergy may result in CI values of between about 0.9 and 0, for example between 0 and 0.5, for example less than about 0.9, 0.8, 0.7, 0.6, 0.5, 0.4, 0.3, 0.2, or 0.1 and greater than 0.01, 0.05, 0.1, 0.2, 0.3, 0.4, 0.5, 0.6, 0.7, 0.8, or 0.9.

Statistical analysis: GraphPad Prism V8 software was used to make the graphs and the statistical analyses. We used t-tests or one-way ANOVAs followed by appropriate post-hoc tests to determine if the experimental groups were significantly different. Data of the mouse xenograft studies were analyzed by two-way/mixed model ANOVA followed by appropriate post-hoc tests.

### Example 1—Inhibiting MCL1 B16 Mouse Melanoma Cells In Vitro

The ability of MCL1 inhibition to inhibit cell growth of B16 cells was investigated in vitro in a B16 mouse cell model of melanoma. MCL1 inhibition by MCL1i did not inhibit cell growth of B16 cells in vitro, showing that MCL1i alone is not sufficient to affect melanoma cells (FIG. 2 panel A).

### Example 2—Inhibiting MCL1 B16 Mouse Melanoma Cells In Vivo

Next, MCL1 inhibition was tested in-vivo, and surprising, MCL1i alone inhibited tumor growth of B16 melanoma cells in vivo (FIG. 2 panel B). Briefly, 2.0×105 B16F10 melanoma cells were injected into the flanks of 7-week-old C57BL/6J mice. MCL1i was then administered to these tumor-bearing mice, retro-orbitally daily at 40 mg/kg, starting at day 6 post-tumor implantation.

Tumor volume was measured every 2 days with digital calipers. The following formula was used to calculate tumor volume: tumor volume (mm3)=(length in mm×width in mm2)/2. Further, the tumors of the same mice in FIG. 2 panel B were used to determine the ability of MCL1 inhibition to alter tumor-infiltrating immune cell populations was investigated in vivo. FIG. 2C shows flow cytometry of tumor-infiltrating immune cells from vehicle-treated control (Veh.) and the 40 mg/kg MCL1i-treated tumors. Surprisingly, as shown in FIG. 2 panel C, MCL1i drastically decreased the frequency of MDSCs while increasing the frequency of CD8+ T cells in the tumors. This data suggests that MCL-1 inhibition boosted anti-tumor immunity in these mice and inhibited tumor growth in vivo.

### Example 3—Combining MCL1i with Anti-PD1 Treatment In Vivo in a B16 Mouse Model of Melanoma

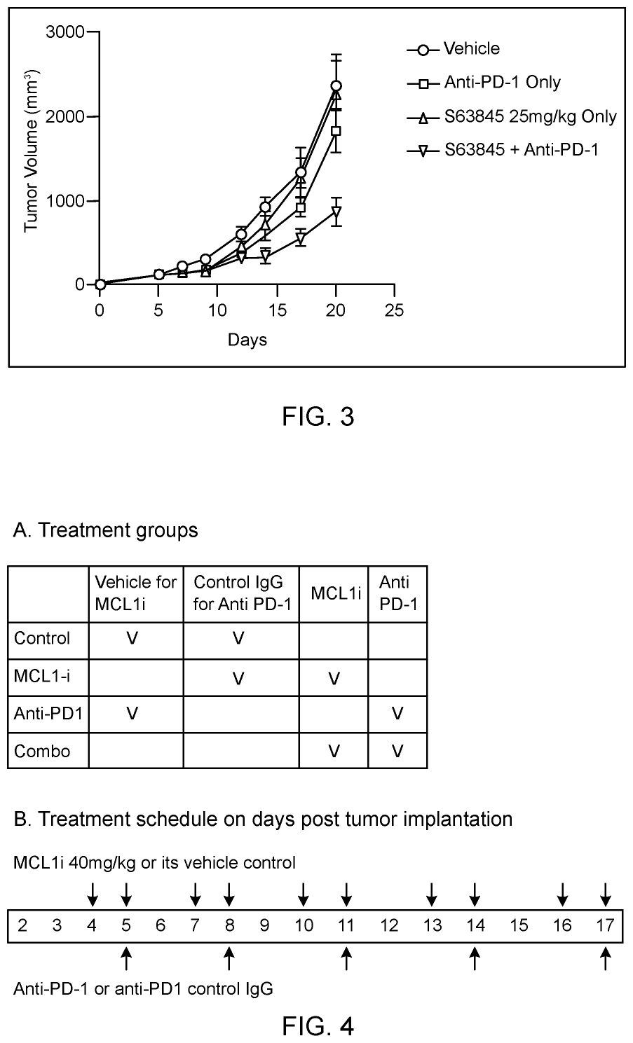

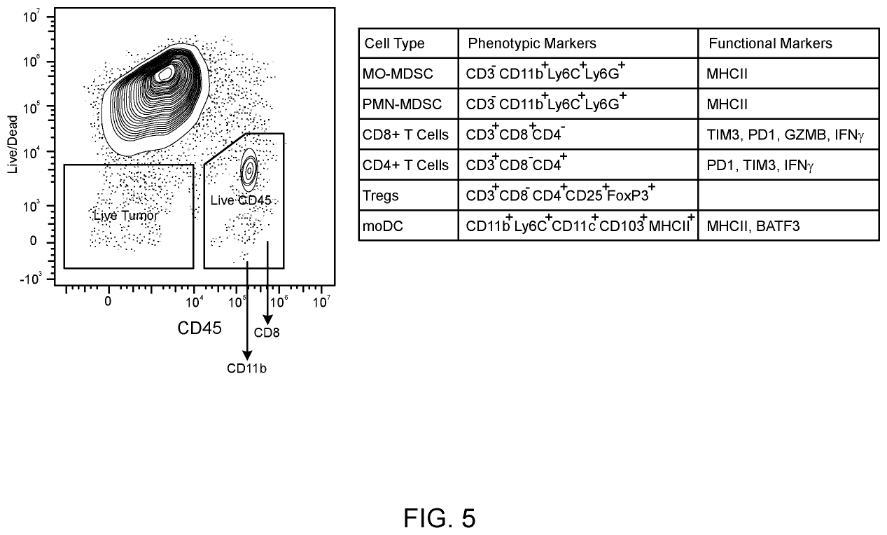

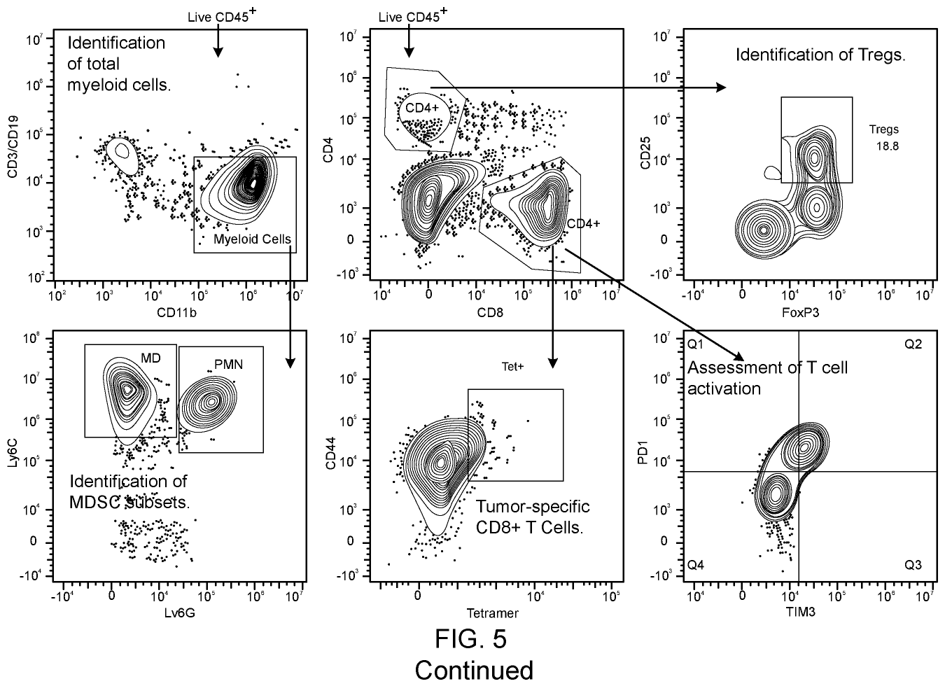

Experiments were performed to determine whether MCL1i increased the efficacy of anti-PD1 therapy. The B16 mouse model of melanoma in BL/6 mice was an appealing experimental model due to the tumor's relative resistance to single agent anti-PD1 therapies. As shown in FIG. 3, combining MCL1i with anti-PD1 strongly suppressed tumor growth, better than either drug alone. These data suggest that MCL1i increases the efficacy of anti-PD1. In this experiment, 2.0×105 B16F10 melanoma cells were again injected into the flanks of 7-week-old C57BL/6J mice. MCL1i was administered at 25 mg/kg on days 5-7, 9, 11 and 12, and anti-PD1 was injected intraperitoneally at 250 μg on days 6, 11, 14 and 16. Tumor volume was measured every 2 days with digital calipers. The following formula was used to calculate tumor volume: tumor volume (mm³)=(length in mm×width in mm²)/2.

### Example 4—TCGA mRNA and Protein Expression Analyses Suggest BCL2 as a Potential Therapeutic Target for BRAF-WT Melanomas

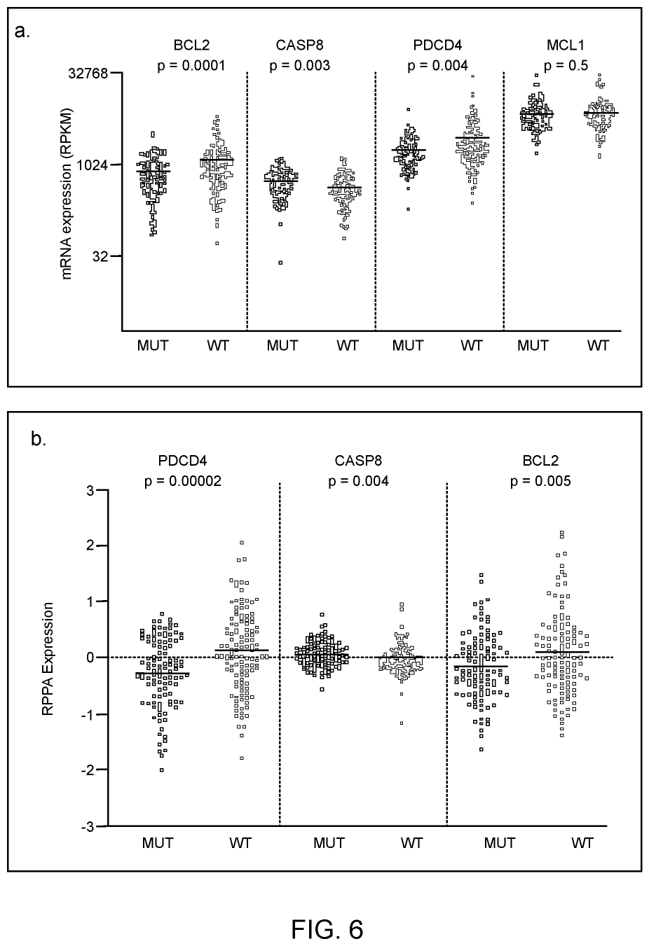

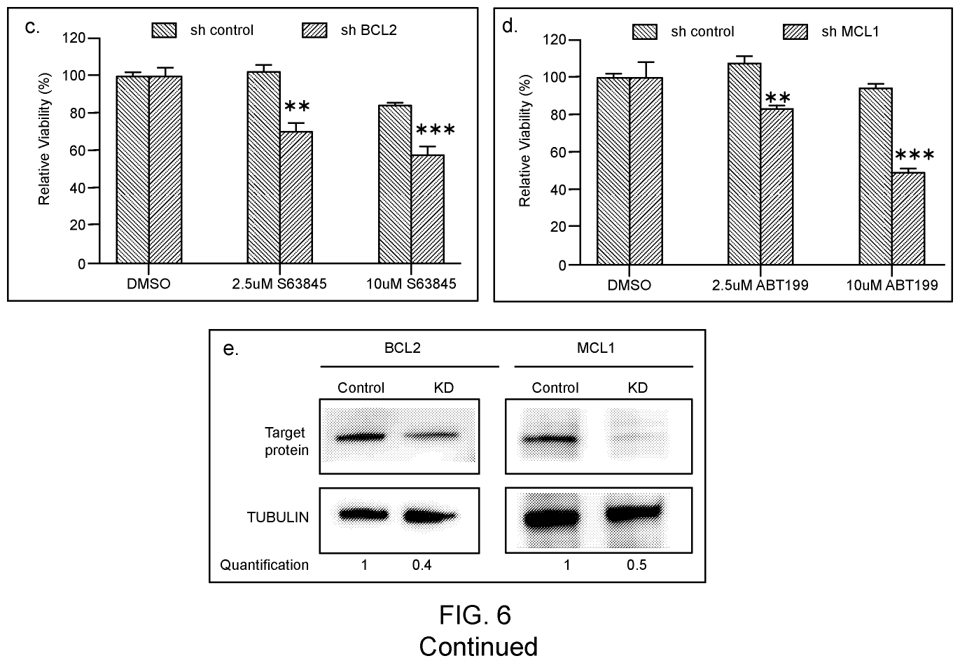

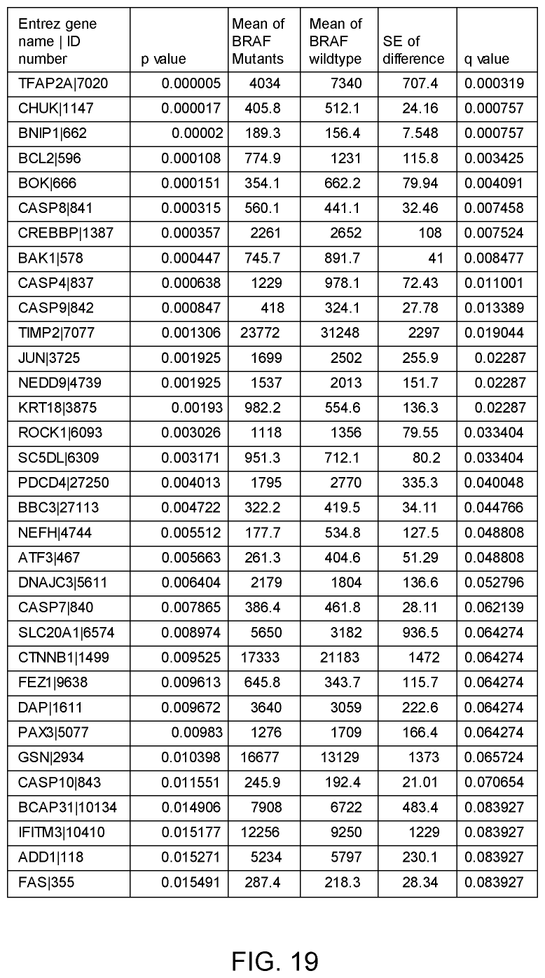

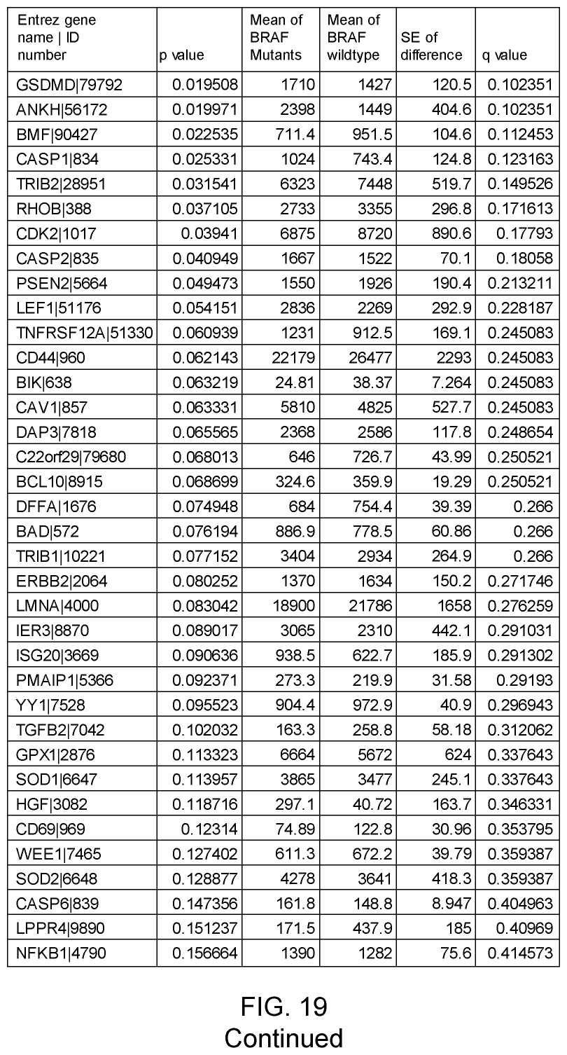

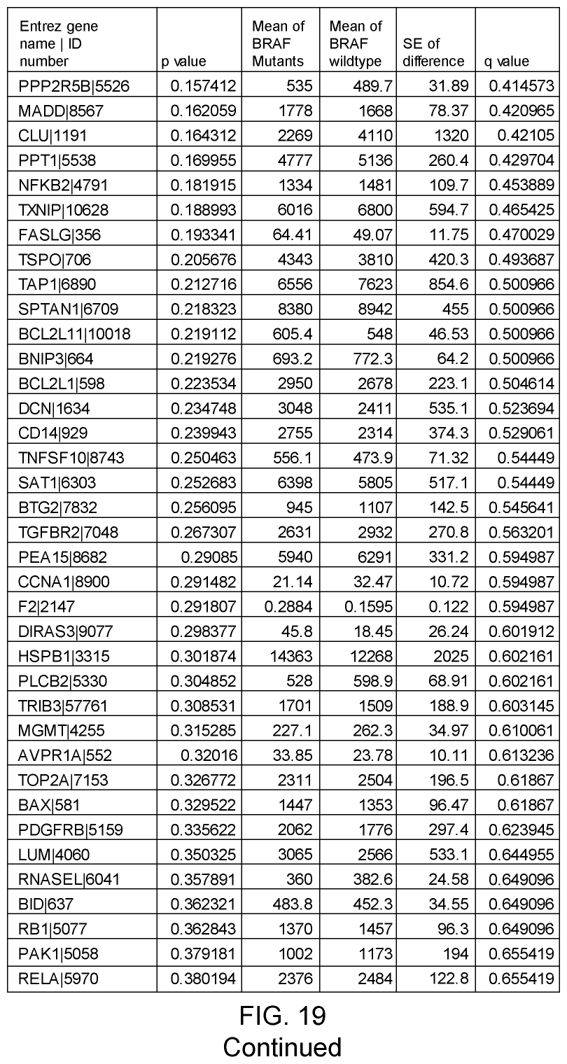

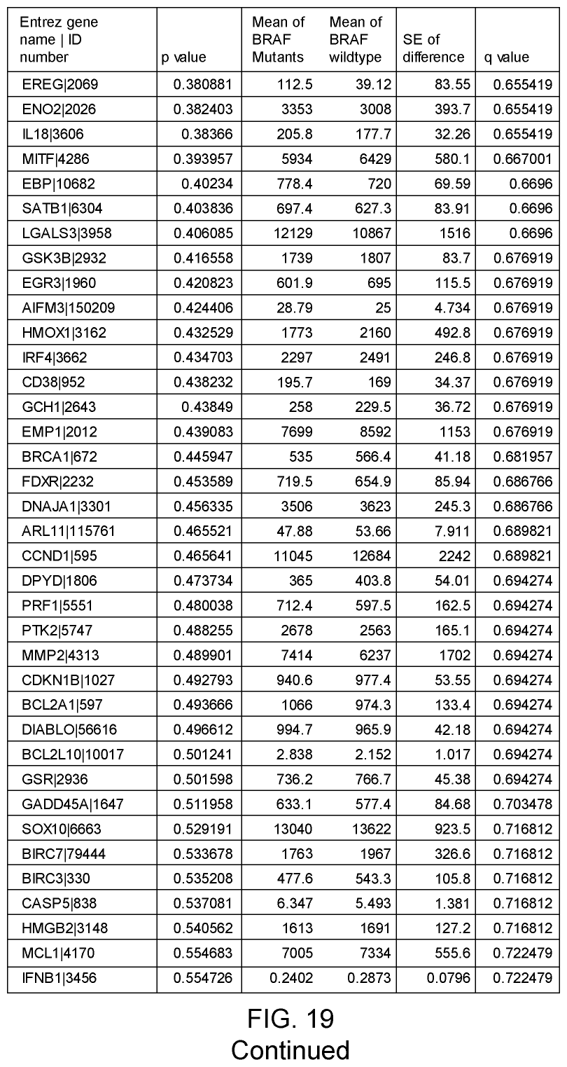

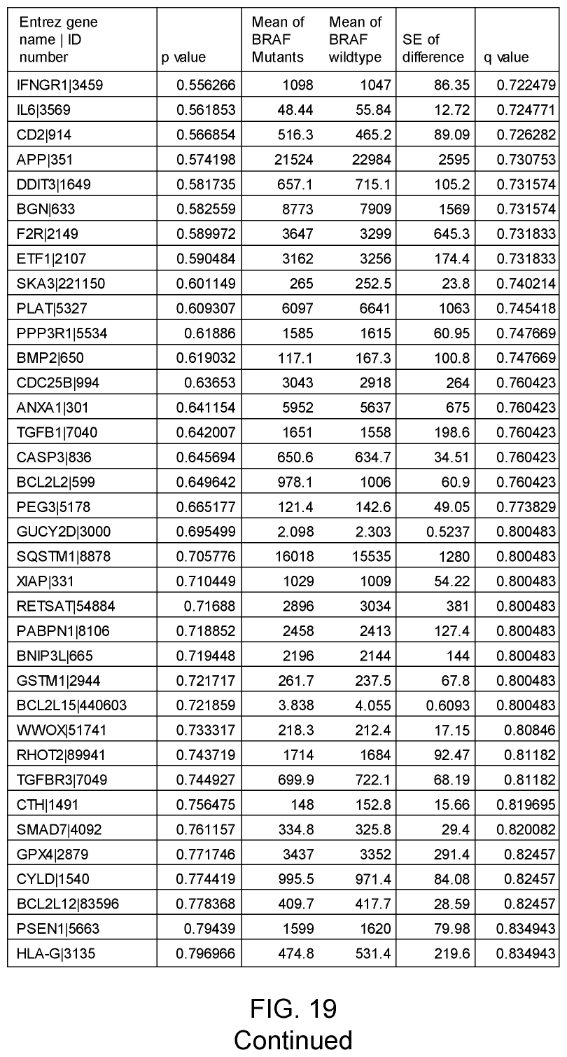

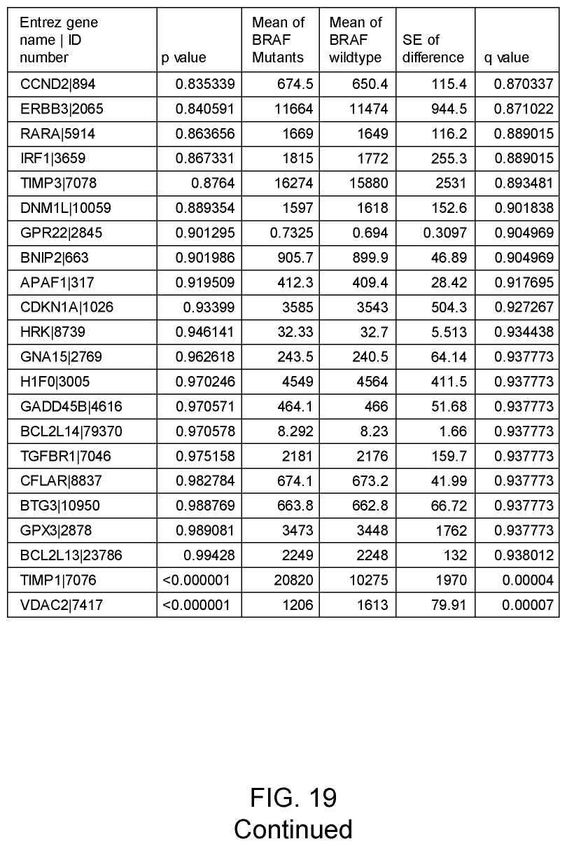

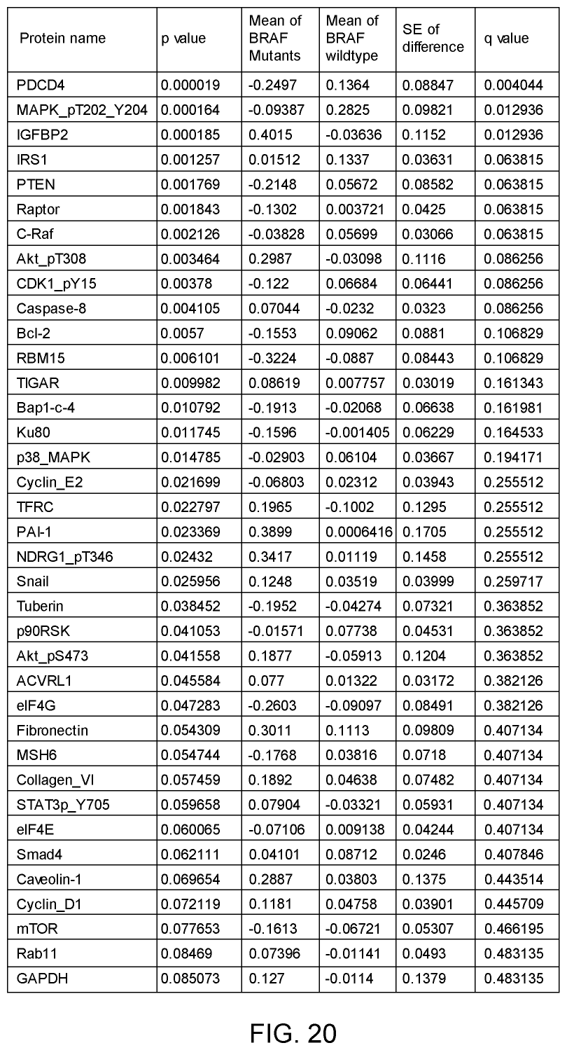

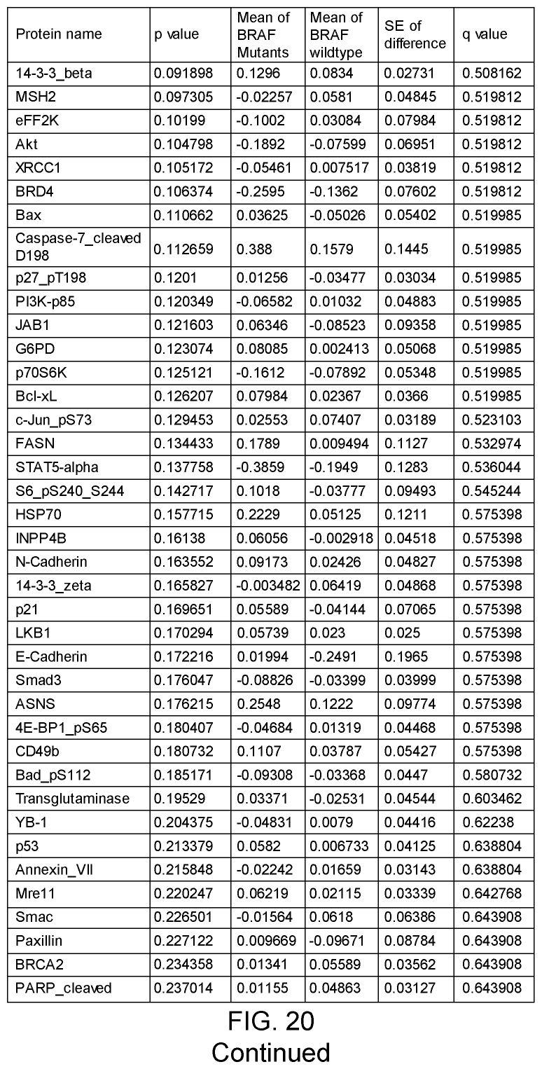

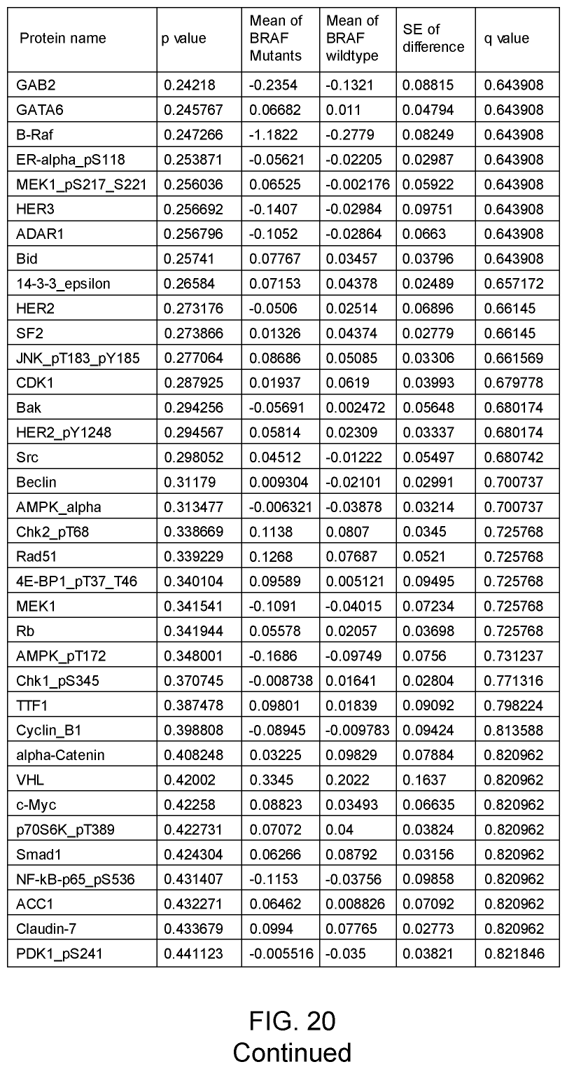

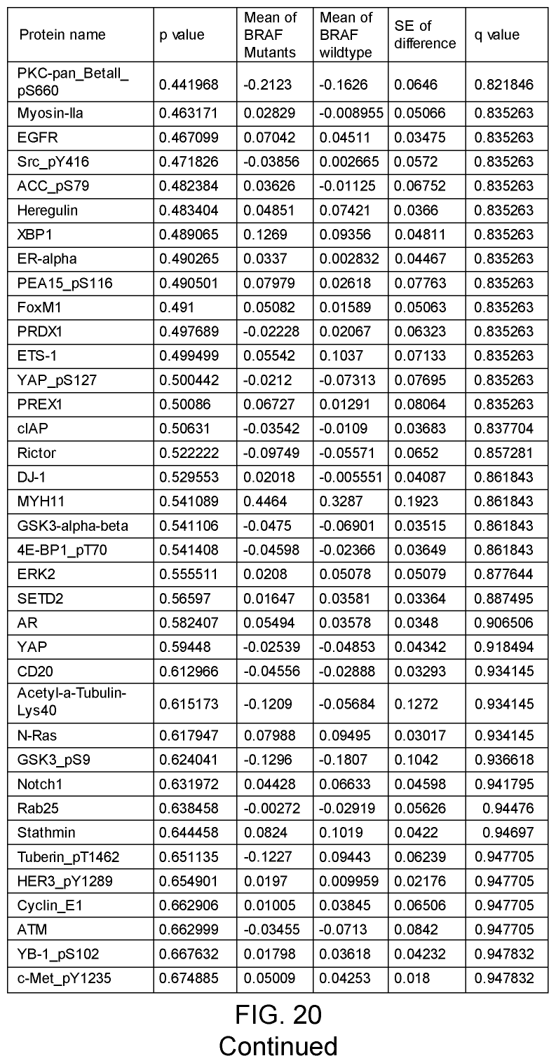

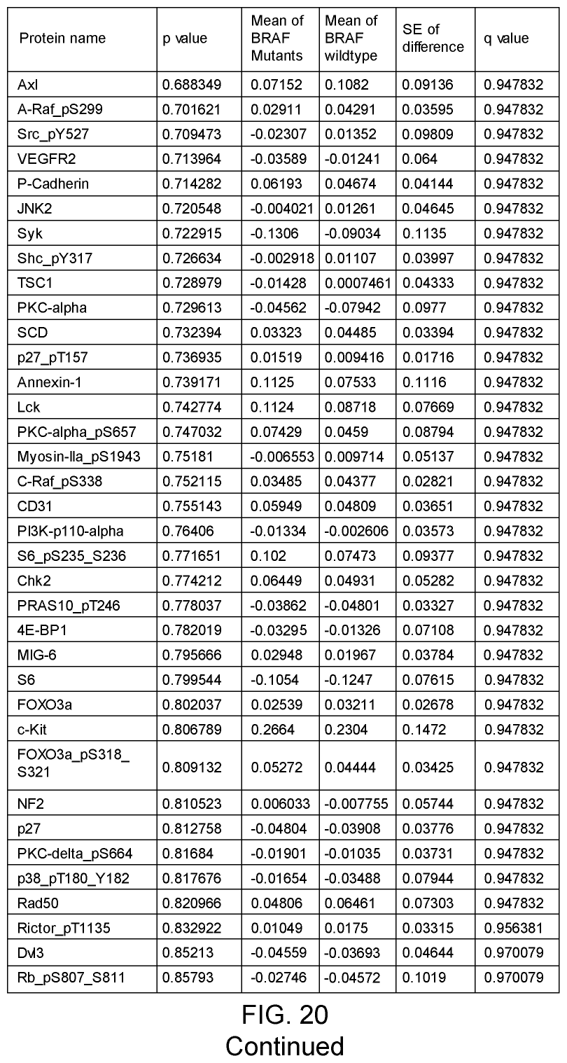

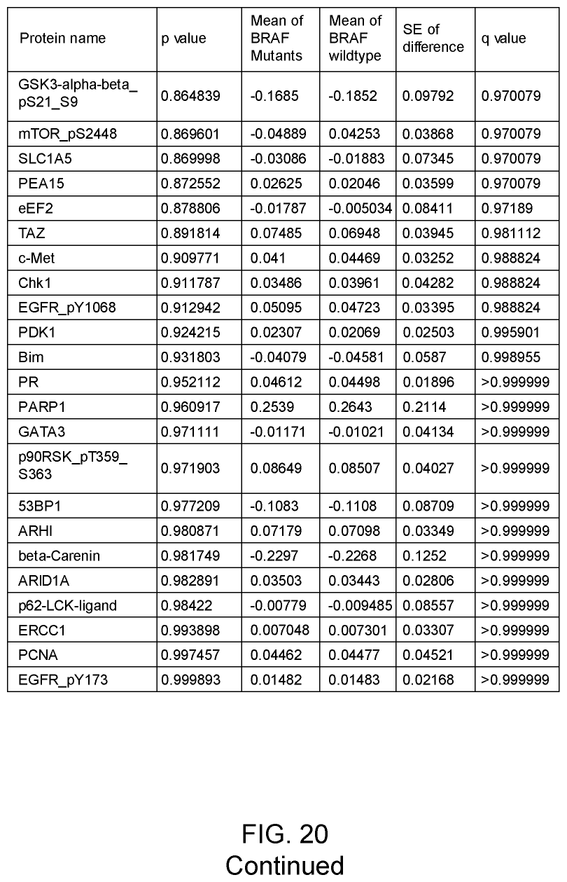

Patients with BRAF-WT melanomas largely lack molecularly targeted treatment options. To identify other potential therapeutic targets, the TCGA data was analyzed for differences in gene and/or protein expression between BRAF-MUT and BRAF-WT melanoma. Samples from the cutaneous melanoma dataset were utilized. Patient groups were defined and classified based on the original parameters set forth in the original TCGA paper, and included samples that had both whole transcriptome (RNA-seq) and reverse phase protein array (RPPA) data available. Using these criteria, 110 samples with a BRAF hotspot mutation and 122 samples with BRAF-WT were identified for further analyses. A targeted approach was taken in analyzing the RNA-seq data and focused on over 200 genes involved in the apoptosis pathway (FIG. 19). The RPPA panel in TCGA was limited and did not include MCL1 (FIG. 20). The only concordant results between these two analyses were higher levels of BCL2 and PDCD4, with a lower level of CASP8 in the WT group, compared with the MUT group (FIG. 6 Panels a and b). Considering that higher BCL2 levels are associated with sensitivity to ABT-199 in other cancers, these data provide a rationale for testing the efficacy of BCL2 inhibitors, such as ABT-199 (venetoclax), in patients with BRAF-WT melanomas.

### Example 5—the Combination of the BCL2 Inhibitor ABT-199 with the MCL1 Inhibitors S63845 has High Efficacy in BRAF-WT Melanomas In Vitro

Work has shown that single agent BH3 mimetics are not effective alone for melanoma, and that MCL1 is an essential anti-apoptotic protein. The combination of MCL1 inhibition with ABT-199 displayed efficacy in neuroblastoma with high BCL2 expression in vitro and in vivo. In melanoma, knocking down BCL2 sensitized cells to the MCL1 inhibitor S63845, and conversely knocking down MCL1 sensitized cells to the BCL2 inhibitor ABT-199 (FIG. 6 Panel c-e). Thus, these results suggest that the simultaneous targeting of both BCL2 and MCL1 is an effective combination to kill melanoma.

The treatment efficacy of combining MCL1 inhibitors with ABT-199 was tested in melanomas with or without BRAF-V600 hotspot mutations (MUT vs WT groups). A panel of patient-derived cell lines was also tested, and these include genetically diverse samples from patients with rare melanoma subtypes (mucosal and acral), and from patients who relapsed from various therapies (Table 1). First, ATP assays were utilized to examine the in vitro viability following the treatments with S63845 and ABT-199, either as a single agent or in combination, in a panel of fifteen human melanoma lines and primary melanocytes (FIG. 7 Panels a-d). FIG. 7 Panel (a) shows a panel of melanomas treated with increasing concentrations of each BH3 mimetic by itself or in combination (Table 2). Overall, single drug treatments of either ABT-199 or S63845 alone (up to 2.5 uM), had little effect on cell viability. Conversely, a reduction in relative viability was observed with combination therapy (FIG. 7 panels a to d). Additionally, there was minimal effect on human primary melanocytes (FIG. 7 panels b). Interestingly, the combination treatment showed a greater efficacy on the BRAF-WT melanomas, as compared to the melanomas with BRAF-V600E (MUT). This similar trend was observed for the combination at a low dose, such as 0.625 uM (FIG. 8). The mean IC50 of the combination was 0.5 uM for BRAF-WT, and the mean IC50 was 1.6 uM, more than 3-fold of IC50 for BRAF-MUT melanomas (FIG. 7 panel c and Table 1). Analyses demonstrate the synergistic effects of this combination, calculated as a combination-index value (FIG. 7 panel d and FIG. 8 panel g). The combination also induced PARP cleavage and caused rounded morphology of cells (FIG. 7 Panels e and f), indicating the induction of apoptosis. Taken together, these data indicate that this combination was effective to kill a wide range of melanomas, however this is more potent in BRAF-WT samples than with BRAF-MUT samples.

TABLE 1
Details of the melanoma lines used in the study and IC50 of indicated drugs.

| | | IC50 | IC50 | IC50 | | Response status to |
|---|---|---|---|---|---|---|
| Cell/MB | | S63845 | ABT-199 | S63845 + ABT- | | current standard of |
| Line | Genotype | (μM) | (μM) | 199 (μM) | Subtype | care |
| MB2114 | BRAF V600E | >10 | >10 | 1.886 | Unknown | BRAFi resistant |
| | | | | | primary | |
| A375 | BRAFV600E | 6.335 | >10 | 1.871 | Cutaneous | N/A |
| 1205Lu | BRAFV600E | 7.195 | >10 | 1.414 | Cutaneous- | N/A |
| | | | | | Metastatic | |
| SKMEL-28 | BRAFV600E | 4 | >10 | 1.271 | Cutaneous | N/A |
| MB2724 | Triple WT (WT for | 3.242 | >10 | 0.6864 | Acral | N/A |
| | BRAF, NRAS and | | | | | |
| | NF-1) | | | | | |
| MB1692 | AGK-BRAF | 7.415 | >10 | 0.637 | Superficial | PD1 responder |
| | | | | | spreading | |
| MB2141 | EML4-ALK | 2.67 | >10 | 0.6286 | Mucosal | CTLA4 responder, |
| | | | | | | treatment stopped |
| | | | | | | and then had a |
| | | | | | | relapse |
| Hs852T | WT for BRAF | 6.143 | 8.805 | 0.5368 | Cutaneous | N/A |
| MB3429 | BRAF G596C, | 8.854 | 3.91 | 0.5368 | Nodular | PD1 partial |
| | GNA11 R183C | | | | | response |
| MB4667 | NRAS Q61R | >10 | 7.69 | 0.527 | Acral | Immunotherapy |
| | | | | | | relapsed |
| Hs294T | WT for BRAF/NRAS | 2.901 | >10 | 0.485 | Cutaneous | N/A |
| MB3616 | NRAS Q61K | 8.107 | 9.69 | 0.452 | Superficial | N/A |
| | | | | | spreading | |
| MB3961 | NRAS Q61K | 2.907 | >10 | 0.4539 | Nodular | Immunotherapy |
| | | | | | | relapsed |
| MB3447 | Triple WT (WT for | 5.49 | >10 | 0.348 | Superficial | N/A |
| | BRAF, NRAS and | | | | spreading | |
| | NF-1) | | | | | |
| WM852c | NRAS | 7.811 | >10 | 0.145 | Cutaneous | N/A |

TABLE 2
p values for ATP assay of S63845 +
ABT-199 Combination (FIG. 7a).

| | | |
|---|---|---|
| DMSO | ABT-199 | S63845 |
| vs Combo | vs Combo | vs Combo |

Dose of 156 nM

| | | | |
|---|---|---|---|
| MB3447 | <0.0001 | <0.0001 | <0.0001 |
| MB3429 | <0.0001 | <0.0001 | <0.0001 |
| MB4667 | <0.0001 | 0.0002 | <0.0001 |
| MB2114 | ns | ns | ns |

Dose of 625 nM

| | | | |
|---|---|---|---|
| MB3447 | <0.0001 | <0.0001 | <0.0001 |
| MB3429 | <0.0001 | <0.0001 | <0.0001 |
| MB4667 | <0.0001 | <0.0001 | <0.0001 |
| MB2114 | ns | ns | ns |

Dose of 2.5 μM

| | | | |
|---|---|---|---|
| MB3447 | <0.0001 | <0.0001 | <0.0001 |
| MB3429 | <0.0001 | <0.0001 | <0.0001 |
| MB4667 | <0.0001 | <0.0001 | <0.0001 |
| MB2114 | <0.0001 | <0.0001 | <0.0001 |

Dose of 10 μM

| | | | |
|---|---|---|---|
| MB3447 | <0.0001 | <0.0001 | <0.0001 |
| MB3429 | <0.0001 | <0.0001 | <0.0001 |
| MB4667 | <0.0001 | <0.0001 | <0.0001 |
| MB2114 | <0.0001 | <0.0001 | <0.0001 |

The numbers indicate the p values. ns denotes not significant.

### Example 6—the Combination of ABT-199 with S63845 Effectively Slowed Tumor Growth In Vivo

The in vivo efficacy of the combination therapy was evaluated in a mouse xenograft model of MB 3616, which has a NRAS-Q61K mutation and does not have a BRAF-V600E/K mutation (FIG. 8 Panels a-b). The combination treatment significantly inhibited tumor growth when compared to the control or single drug treatments (p<0.001) (FIG. 8 Panel a). There were no significant changes in mouse weight (FIG. 8 Panel b), suggesting no obvious adverse effects. Immunohistochemistry for Cleaved Caspase 3 (an apoptosis marker) and Ki67 (a proliferation marker) on the tumor sections showed that the combination treatments significantly decreased Ki67 positive cells (p<0.001) and increased the Cleaved Caspase 3 positive cells (p<0.05) (FIG. 8 Panels c, d and e).

Current in vitro data suggested that the combination of MCL1 inhibitor and ABT-199 was effective in BRAF-MUT melanomas, but at higher concentrations of the compounds than the BRAF-WT genotype needed (FIG. 7, FIG. 8 Panels f and j). In vivo, this combination was also successful in inhibiting tumor growth for BRAF-V600E melanoma, when ABT-199 was administered at an increased frequency of three times per week (FIG. 8 Panel h). There was minimal toxicity in our mouse xenograft model. Even with the higher frequency of dosing, there were no significant changes in mouse weight (FIG. 8 Panel i) and the mice were healthy overall. These results collectively indicate that the combination of the MCL1 inhibitors with the BCL2 inhibitor ABT-199 is effective in killing advanced melanomas.

### Example 7—the Combination of ABT-199 with S63845 Significantly Inhibited Sphere-Forming Capacity of the Melanoma Initiating Cells

In melanoma, a sub-population of cells has enhanced plasticity, drug resistance and stem-cell-like features. These cells are referred to as Melanoma Initiating Cells (MICs) and may contribute to drug resistance and relapse. Although ABT-199 has been shown to kill leukemia stem cell populations, this effect has not been reported for MICs. Two commonly used methods were employed, primary and secondary sphere forming assays. The primary sphere assay enriches MIC populations, and can be used to measure the effects of drug treatments on MIC populations, whereas the secondary sphere assay measures the self-renewal capacity of the MICs after initial treatment.

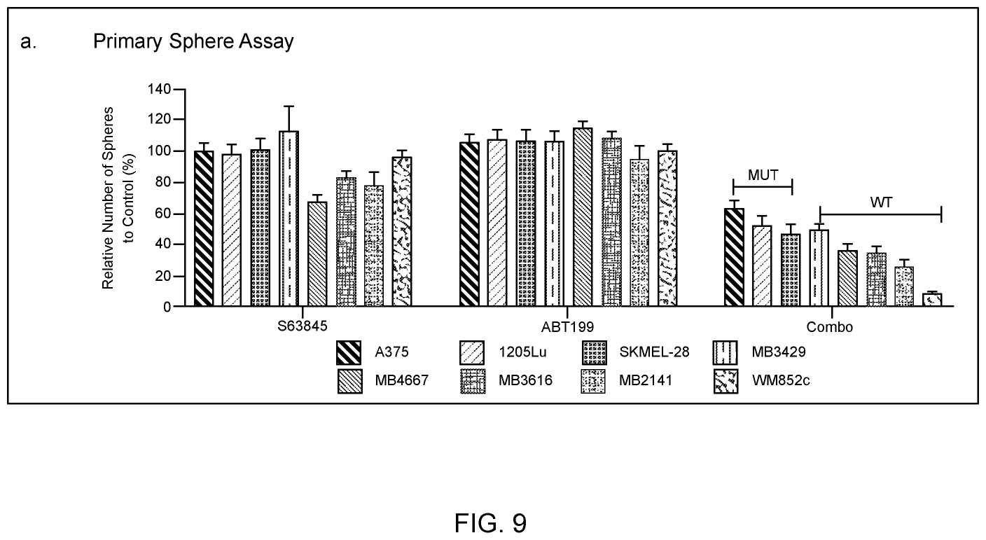

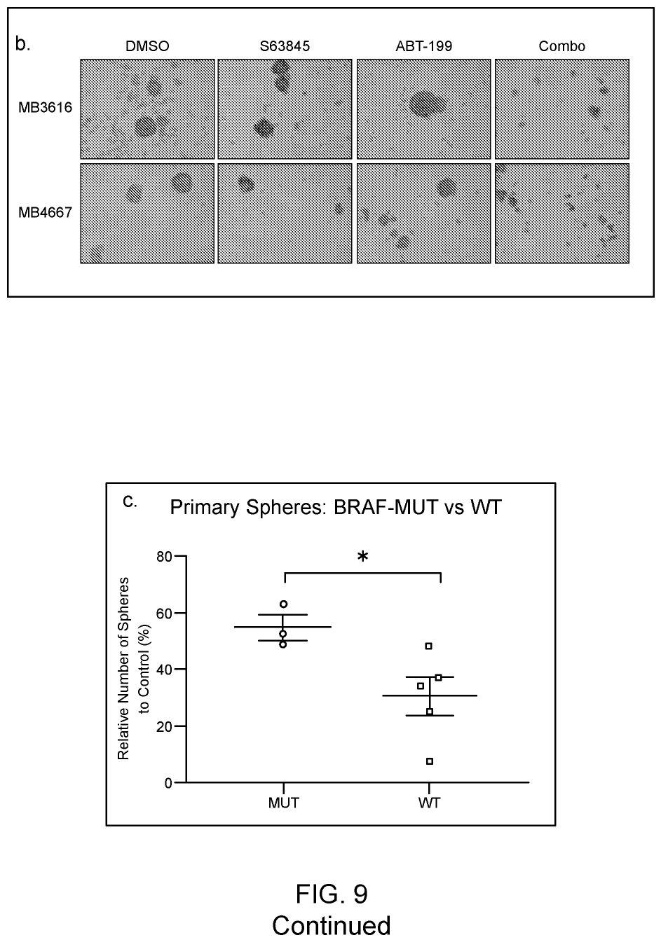

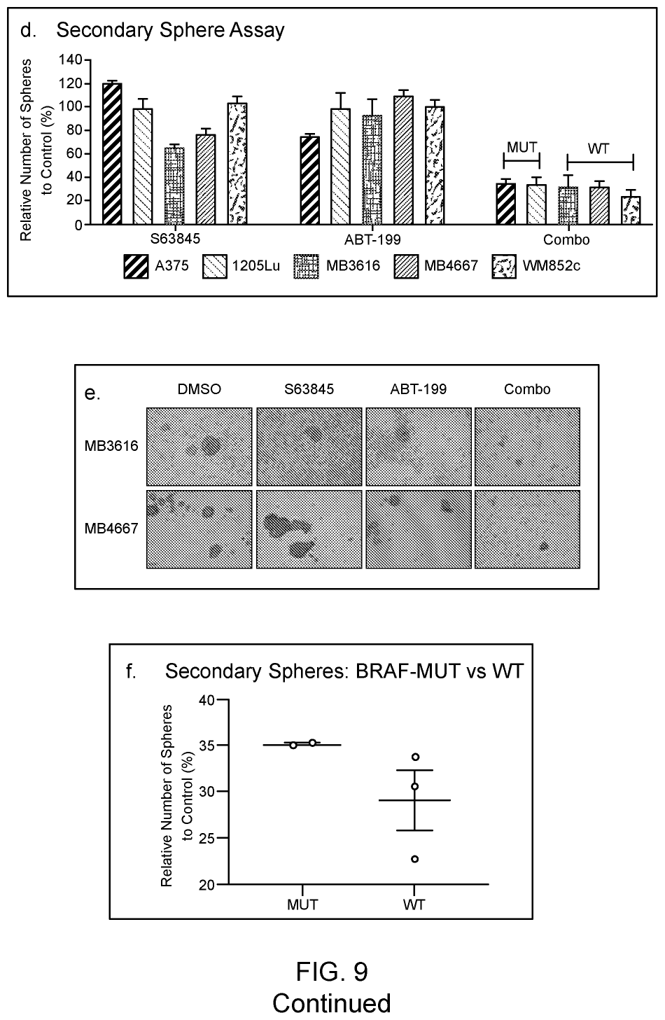

The combination therapy significantly disrupted primary spheres (FIG. 9 Panels a and b, Table 3) and eliminated almost all sphere formation in the secondary sphere assays, compared to DMSO or single drug treatment (p<0.001) in all cell lines tested (FIG. 9 Panels d and e, Table 3), suggesting the combination was effective to kill MICs and inhibited self-renewal capacity. Similar to the ATP assay (FIG. 7 panel c), the combination was more successful in inhibiting the primary spheres in lines with BRAF-WT (WT) genotypes than those with BRAF-V600E (MUT) (FIG. 9 panel c). There was no significant difference in secondary sphere formation ability between the BRAF MUT vs WT lines (FIG. 9 panel f). These results suggest that the combination of ABT-199 and S63845 can play an important role in preventing relapse caused by MICs.

TABLE 3
p values for FIG. 9.

| | | |
|---|---|---|
| DMSO | ABT-199 | S64315 |
| vs Combo | vs Combo | vs Combo |

P values for Primary Sphere Assay (FIG. 9 Panel (a))

| | | | |
|---|---|---|---|
| A375 | 0.0061 | 0.0025 | 0.0053 |
| 1205Lu | 0.0016 | 0.0006 | 0.0019 |
| SKMEL-28 | 0.0036 | 0.0016 | 0.0029 |
| MB3429 | 0.0253 | 0.0130 | 0.0069 |
| MB4667 | <0.0001 | <0.0001 | 0.0021 |
| MB3616 | <0.0001 | 0.0004 | <0.0001 |
| MB2141 | 0.0001 | 0.0001 | 0.0010 |
| WM852c | 0.0036 | 0.0016 | 0.0029 |

P values for Secondary Sphere Assay (FIG. 9 panel (d))

| | | | |
|---|---|---|---|
| A375 | 0.0002 | 0.0057 | <0.0001 |
| 1205Lu | 0.0030 | 0.0030 | 0.0035 |
| MB3616 | 0.0030 | 0.0059 | 0.0277 |
| MB4667 | <0.0001 | <0.0001 | 0.0009 |
| WM852c | <0.0001 | <0.0001 | <0.0001 |

The numbers indicate the p values.

### Example 8—the Effects of ABT-199+S63845 is Partially Dependent on Pro-Apoptotic BCL2 Family Members NOXA, BIM, and BID

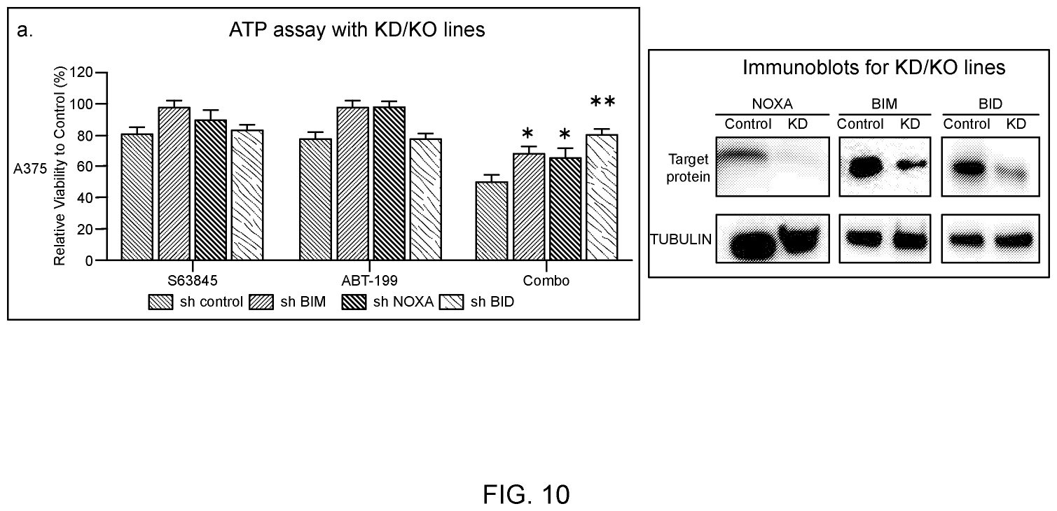

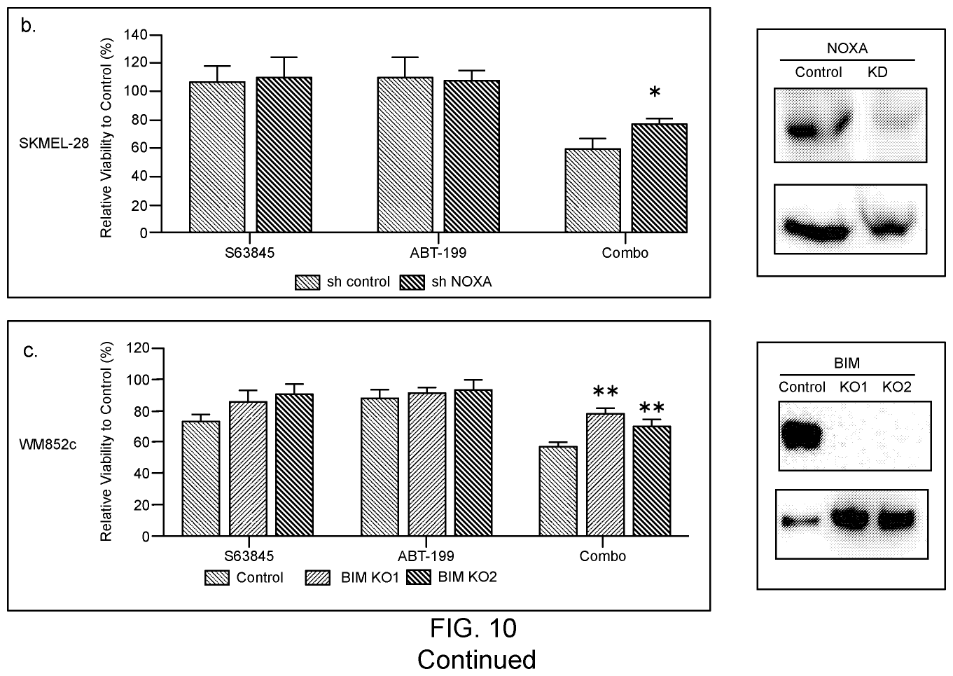

The NOXA-BIM-MCL1 axis plays a crucial role in BH3 mimetic induced cell death. In B cell lymphoma cells, genomic amplification or pharmacologic induction of NOXA sensitizes cells to BCL2 inhibitors, including ABT-199. In mantle cell lymphoma, both BIM and NOXA mediate ABT-199-induced cell death. In acute myeloid leukemia (AML), BIM is an important mediator for S63845-induced apoptosis. In melanoma, NOXA and BIM mediate the killing effects of several combinations, including the BH3 mimetic ABT-737 and ABT-263. Thus, the roles of BIM and NOXA were investigated in the S63845+ABT-199 mediated cell death with cell lines genetically modified with shRNA or CRISPR-Cas9 technology. Knocking down or knocking out BIM or NOXA partially protected melanoma cells from the combination treatment but did not eliminate the killing effects (FIG. 10 Panels a-c).

### Example 9—S64315 has Similar Synergistic Effects as S63845, when Combined with ABT-199

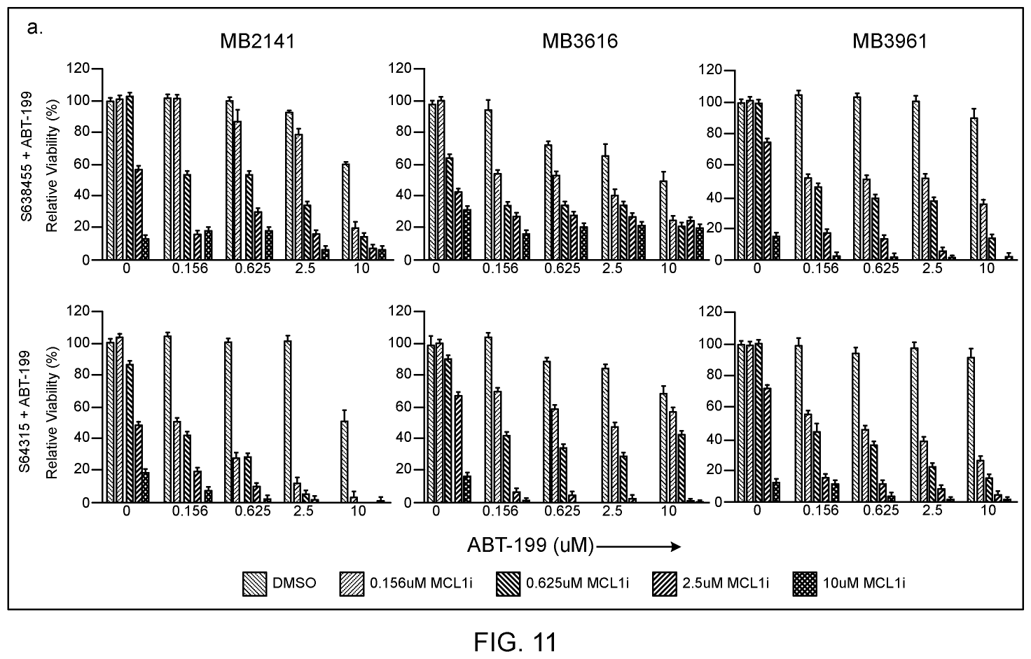

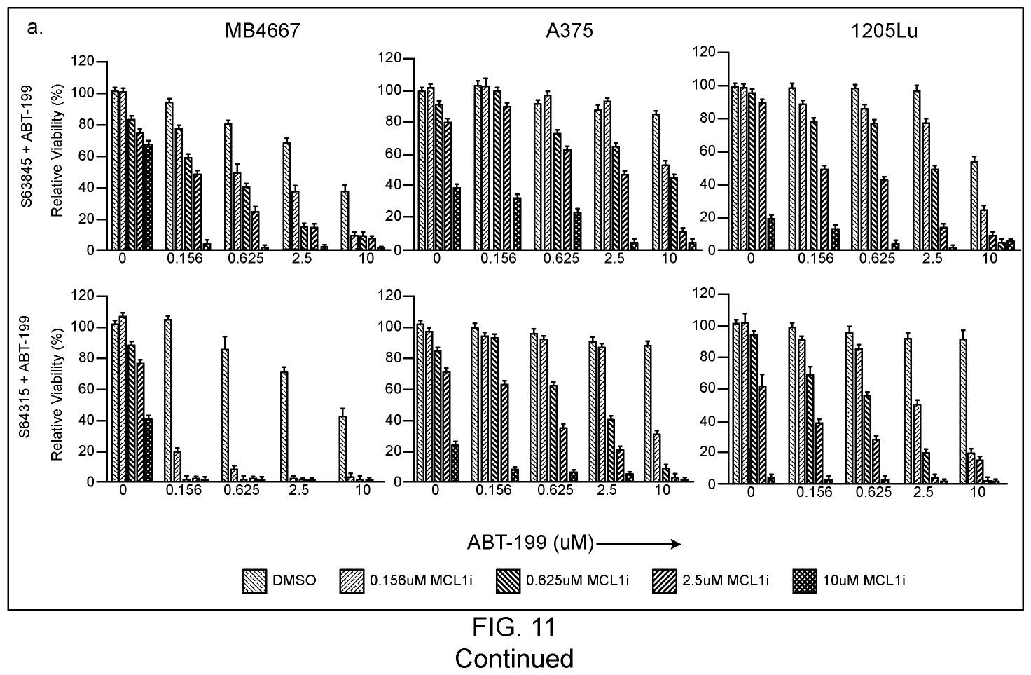

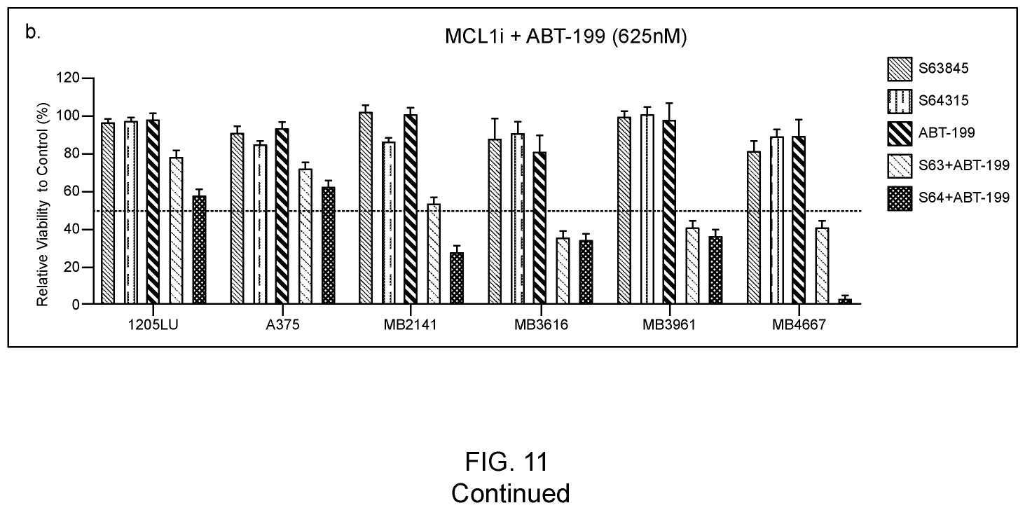

S64315 (MIK665), which is derived from S63845, has similar chemical properties to inhibit MCL1, and is currently in clinical trials for AML (ClinicalTrials.gov NCT02992483; NCT02979366). Thus, a comparative analysis of S64315 and S63845 was performed, either alone or in combination with ABT-199 in multiple melanoma cell lines. These include both BRAF-WT and BRAF-MUT lines. Overall, S64315 exhibited similar or better efficacy than S63845 (FIG. 11, Table 4).

TABLE 4
p values for FIG. 11.

| | | |
|---|---|---|
| DMSO | ABT-199 | S64315 |
| vs Combo | vs Combo | vs Combo |

Dose of 156 nM

| | | | |
|---|---|---|---|
| MB2141 | <0.0001 | <0.0001 | <0.0001 |
| MB3616 | <0.0001 | <0.0001 | <0.0001 |
| MB3961 | <0.0001 | <0.0001 | <0.0001 |
| MB4667 | <0.0001 | <0.0001 | <0.0001 |
| A375 | ns | ns | ns |
| 1205Lu | ns | 0.0138 | ns |

Dose of 625 nM

| | | | |
|---|---|---|---|
| MB2141 | <0.0001 | <0.0001 | <0.0001 |
| MB3616 | <0.0001 | <0.0001 | <0.0001 |
| MB3961 | <0.0001 | <0.0001 | <0.0001 |
| MB4667 | <0.0001 | <0.0001 | <0.0001 |
| A375 | <0.0001 | <0.0001 | <0.0001 |
| 1205Lu | <0.0001 | <0.0001 | <0.0001 |

Dose of 2.5 μM

| | | | |
|---|---|---|---|
| MB2141 | <0.0001 | <0.0001 | <0.0001 |
| MB3616 | <0.0001 | <0.0001 | <0.0001 |
| MB3961 | <0.0001 | <0.0001 | <0.0001 |
| MB4667 | <0.0001 | <0.0001 | <0.0001 |
| A375 | <0.0001 | <0.0001 | <0.0001 |
| 1205Lu | <0.0001 | <0.0001 | <0.0001 |

Dose of 10 μM

| | | | |
|---|---|---|---|
| MB2141 | <0.0001 | <0.0001 | <0.0001 |
| MB3616 | <0.0001 | <0.0001 | <0.0001 |
| MB3961 | <0.0001 | <0.0001 | 0.0196 |
| MB4667 | <0.0001 | <0.0001 | <0.0001 |
| A375 | <0.0001 | <0.0001 | <0.0001 |
| 1205Lu | <0.0001 | <0.0001 | ns |

The numbers indicate the p values. ns denotes not significant.

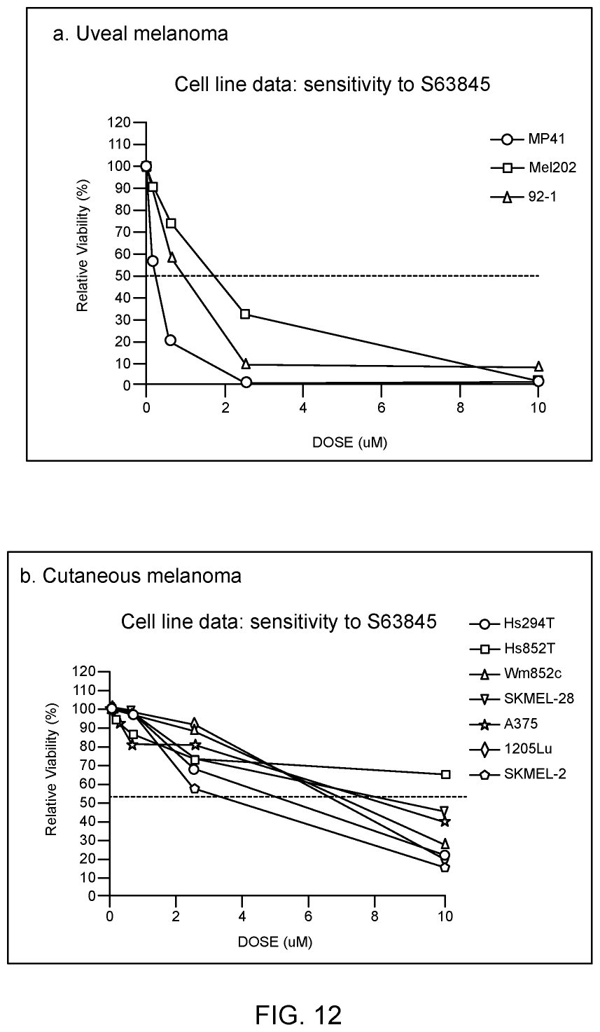

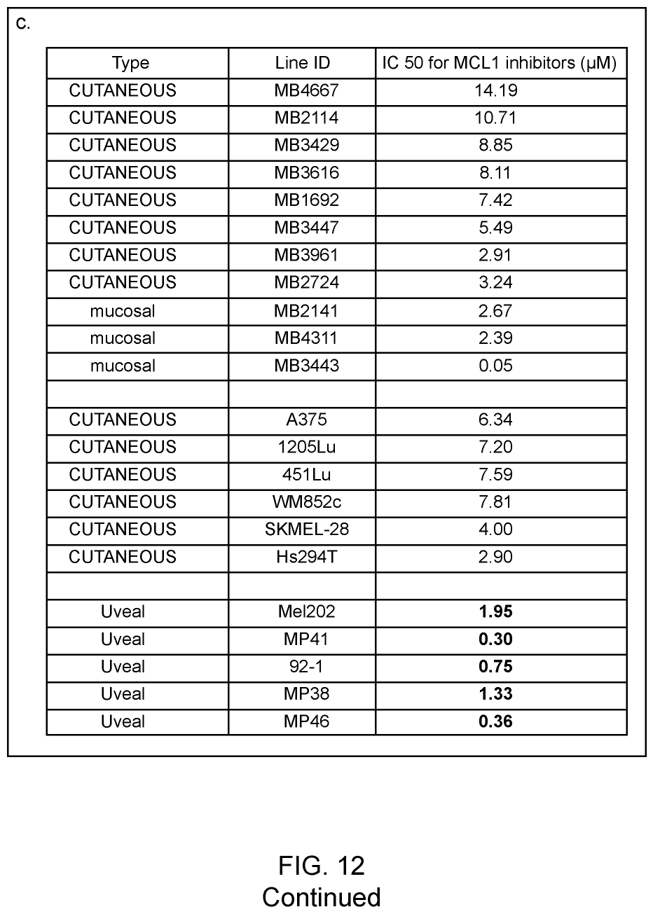

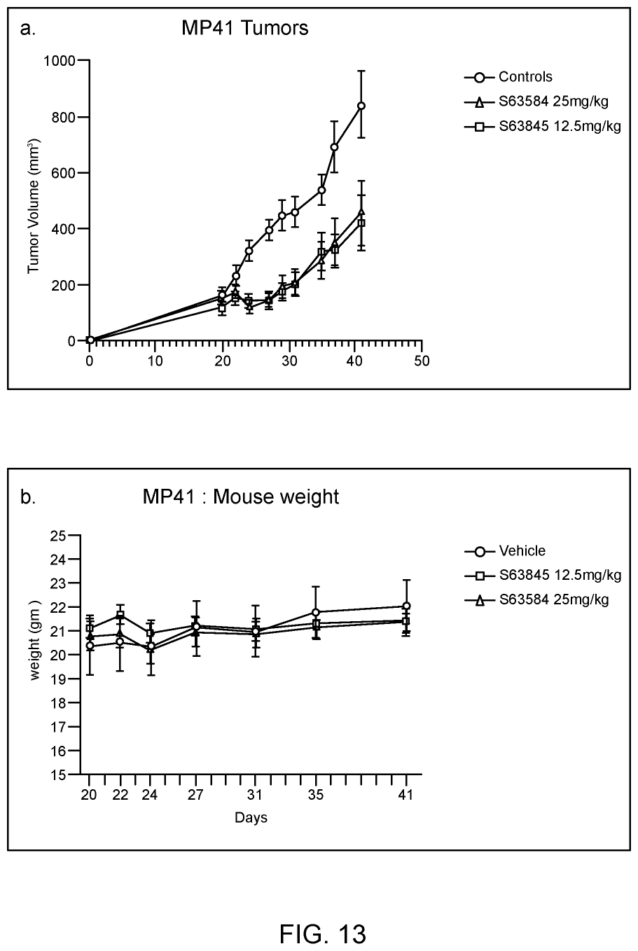

### Example 10—MCL1 Inhibitors Alone are Effective at Killing Uveal Melanoma In Vitro (FIG. 12) and In Vivo (FIG. 13)

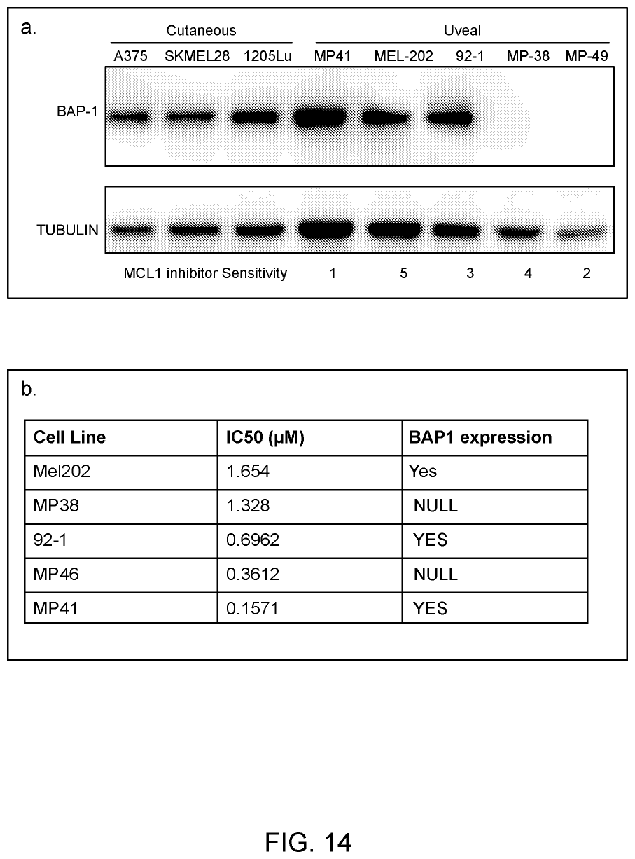

Loss of BAP1 is associated with more aggressive uveal melanoma, and it did not alter sensitivity to MCL1 inhibitors (FIG. 14). These results suggest that MCL1 inhibitors may be effective with even very aggressive uveal melanomas. Some of mucosal melanomas may also be good targets for MCL1 inhibitors, since one of mucosal melanomas, MB3443, was very sensitive to MCL1 inhibitors with IC 50 of 0.05 uM (FIG. 12). It is noteworthy to mention that there is no FDA approved drugs yet for treatment of uveal melanoma.

### Example 11—the Combinations of MCL1 Inhibitors Plus BCL2 Inhibitors are Therapeutic Options for Patients with Advanced Melanoma, Especially for Those without Hotspot BRAF-V600 Mutations

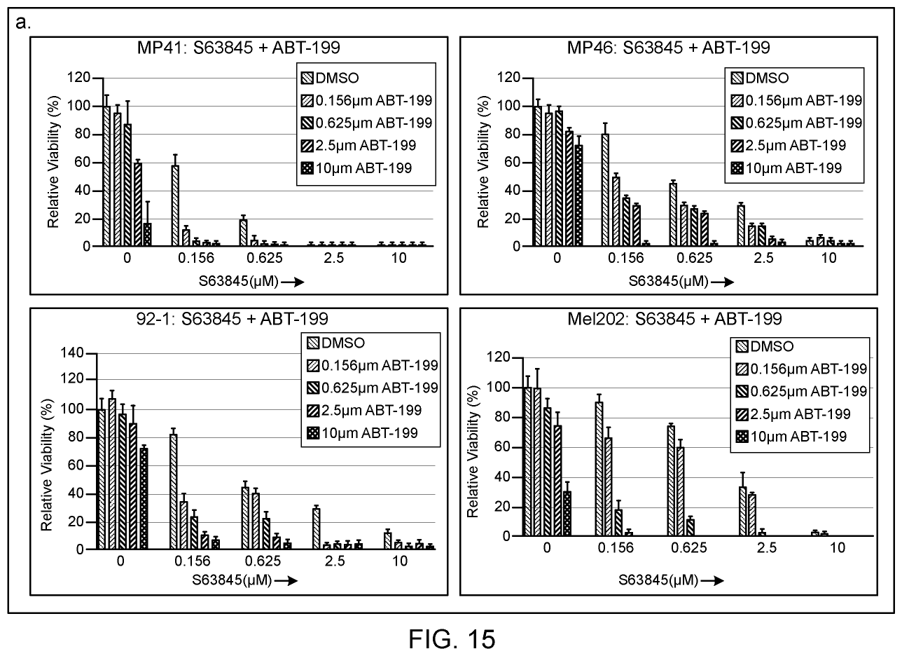

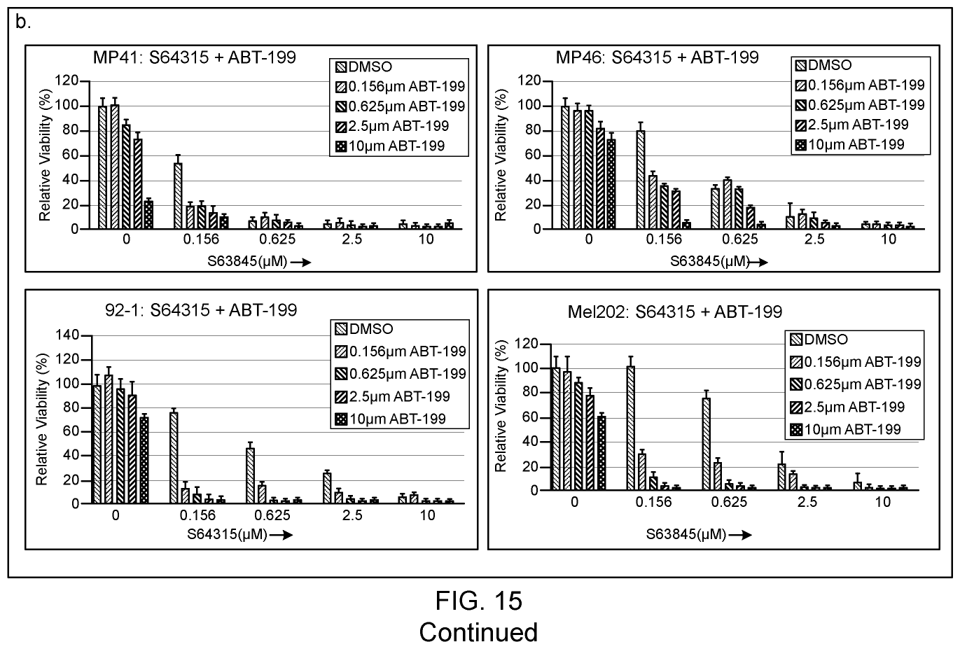

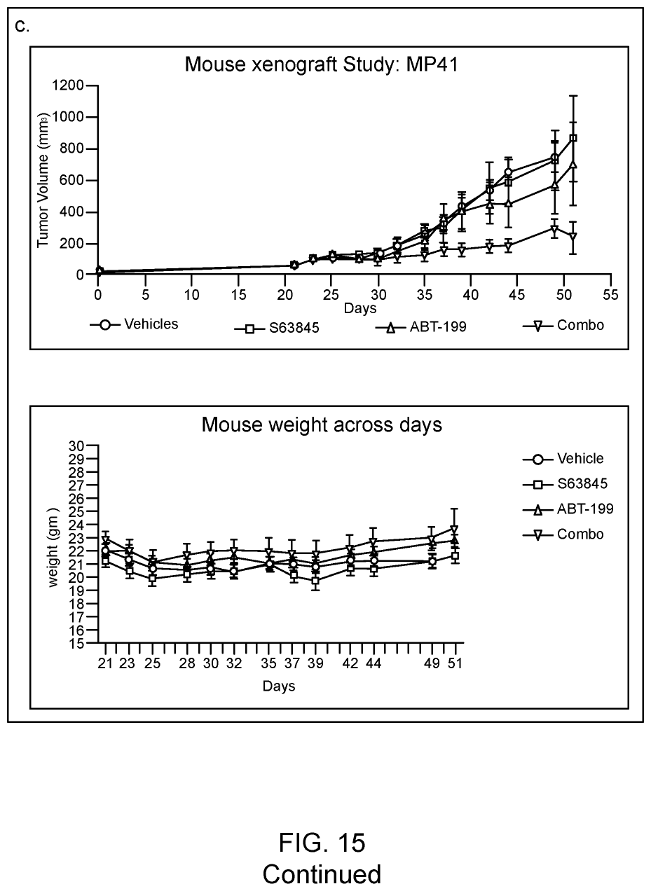

In melanoma, knocking down BCL2 sensitized cells to a MCL1 inhibitor, and knocking down MCL1 sensitized cells to a BCL2 inhibitor (FIG. 6 Panels c and d), suggesting targeting both MCL1 and BCL2 simultaneously is a good option to kill melanomas. In vitro, this combination has high efficacy to kill melanomas, especially for the ones without BRAF-V600E/K mutations (FIG. 7). In vivo, the combinations inhibited tumor growth, better than single drug alone (FIG. 8 Panels a-e and FIG. 15). These include cutaneous melanoma and rare subtypes of uveal, acral and mucosal melanomas.

### Example 12—the Combinations of MCL1 Inhibitors Plus BCL2 Inhibitors Inhibited Sphere-Forming Capacity of Melanoma Initiating Cells

In melanoma, a sub-population of cells has enhanced plasticity, drug resistance and stem-cell-like features. These cells are referred to as Melanoma Initiating Cells (MICs) and may contribute to drug resistance and relapse. Sphere forming assays are commonly used methods to enrich MIC populations. The primary sphere assay measures the MIC populations, whereas the secondary sphere assay measures the self-renewal capacity of the MICs after initial treatment. The combination therapy significantly disrupted primary spheres (FIG. 9 Panels a and b, Table 3) and eliminated almost all secondary sphere formation, compared to DMSO or single drug treatment (p<0.001) in all cell lines tested (FIG. 9 Panels d and e, Table 3), suggesting the combination was effective to kill MICs and inhibited self-renewal capacity. These results suggest that the combination of ABT-199 and S63845 can play an important role in preventing relapse caused by MICs.

### Example 13—the Clinic-Ready Version of MCL1 Inhibitor, S64315, has Comparable or Better Effects than MCL1 Inhibitor S63845 (FIG. 11 Panels a and b)

Combination therapy of S64315/MIK665 (the clinic-ready version of S63845) with ABT-199 has a synergistic effect in treating melanoma samples of diverse genetic backgrounds and is comparable to the effects of S63845 with ABT-199. FIG. 11 Panel a shows ATP assays of melanoma cell lines upon indicated treatments for 48 h. The viability of the DMSO control for each cell line was set to 100%. Both the combinations (S63845+ABT-199; upper panel and S64315+ABT-199, lower panel) had similar efficacy in reducing the cell viability of representative melanoma lines. FIG. 11 Panel b shows the summary of ATP assay data of six melanoma cell lines, including patient derived cell lines, treated with single drug or a combination of S63845+ABT-199 or S64315+ABT-199. All drugs were used at a dose of 625 nM. For visual clarity, the * is not shown in the figures. Both combinations were highly synergistic at sub-micromolar doses. Error bars represent +/−SEM.

### Example 14—MCL1 Inhibitors Enhance Antitumor Immunity and the Efficacy of Immunotherapies for Melanoma

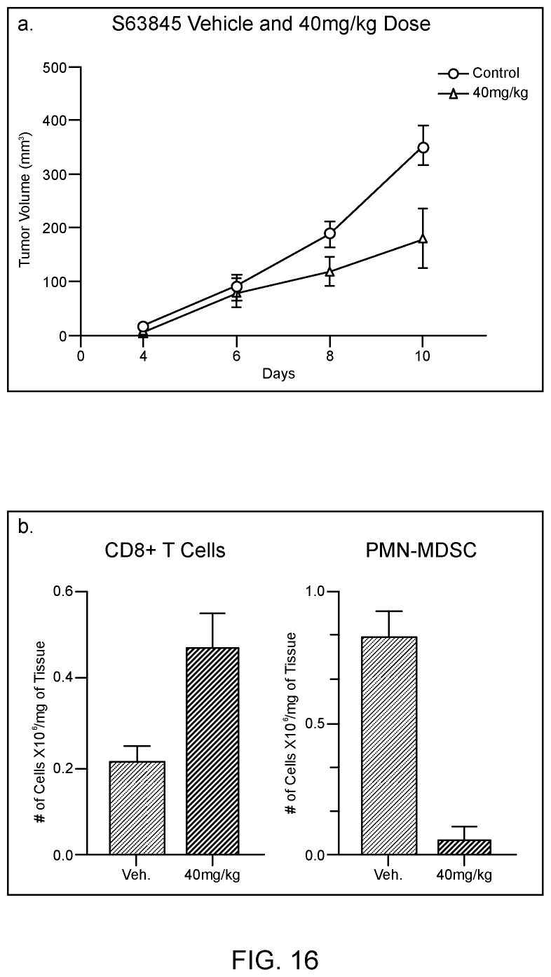

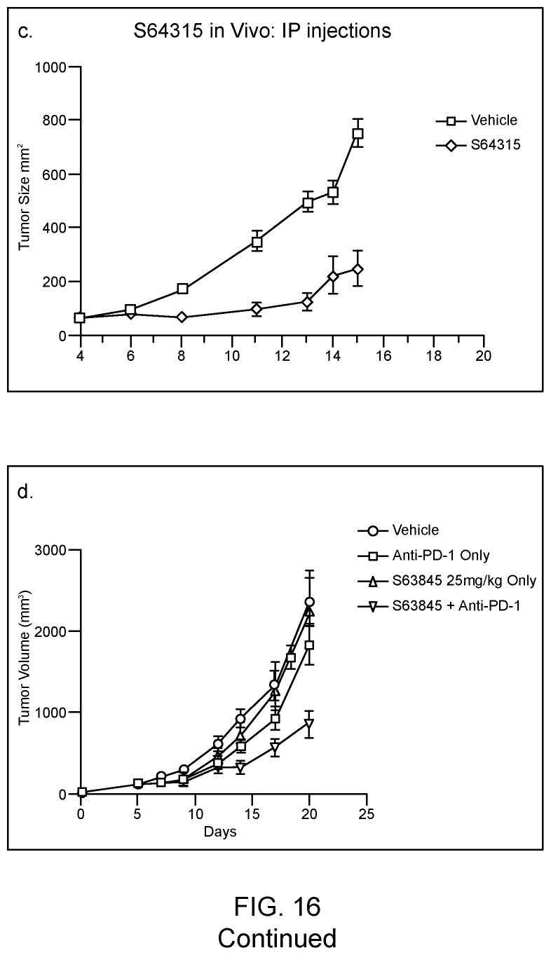

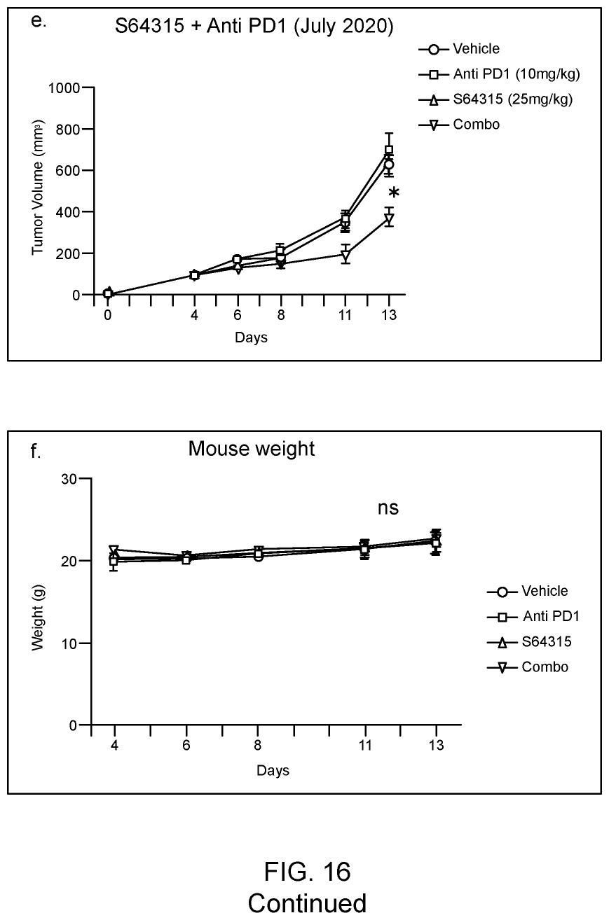

In an immunocompetent mouse model, MCL1 inhibitors S63845 or S64315 blocked tumor growth (FIG. 16 Panels a and c), drastically decreased the frequency of myeloid-derived suppressor cells (MDSCs) and increased the frequency of tumor-infiltrating CD8+ T cells (FIG. 16 Panel b). MDSCs are a heterogeneous population of immature myeloid cells that are a significant source of immunosuppression in tumors. CD8+ T cells, when not immunosuppressed, are tumor-eliminating immune cells. This suggests that BH3 mimetics may tip the balance within the tumor microenvironment toward immune activation and tumor clearance. Furthermore, MCL1 inhibitor increased the efficacy of anti-PD1 treatment on tumor growth in vivo (FIG. 16 Panels d and e).

### Example 15—High-MCL1 Expressing MDSC Cells May Serve as a Biomarker for Adding MCL1 Inhibitor to Immunotherapies

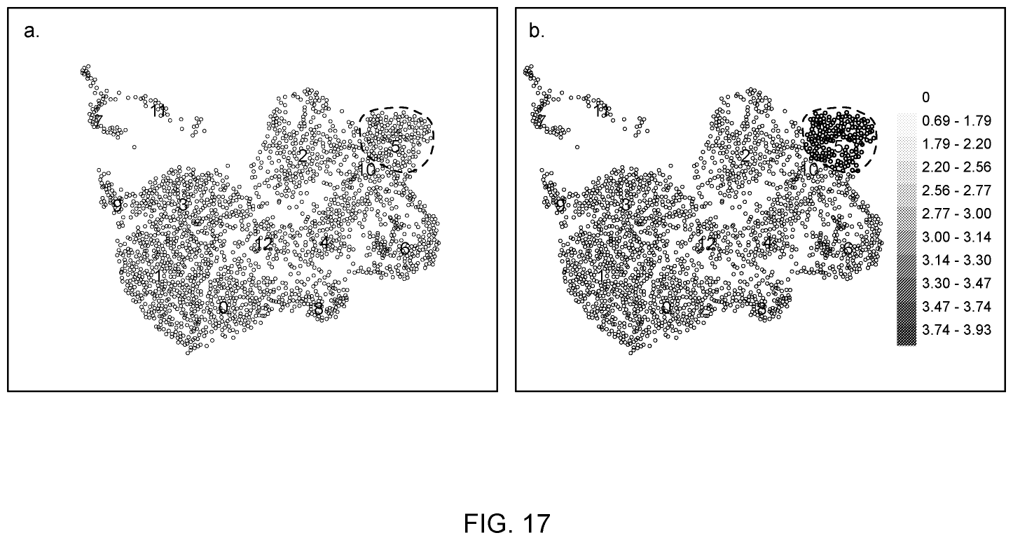

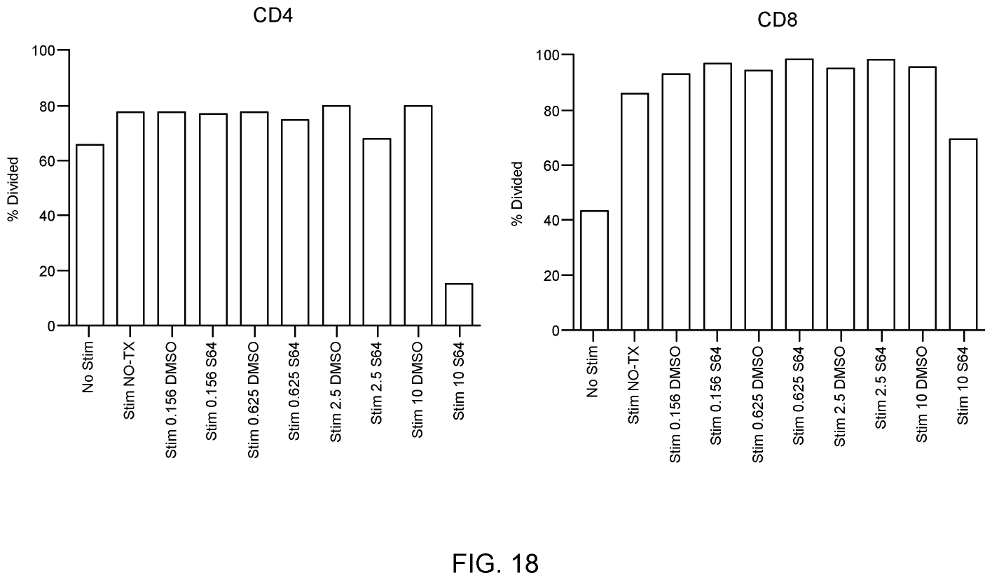

Single-cell RNA-seq of tumor-infiltrating myeloid cells showed high MCL1 expression in the MDSC cell populations from two melanoma patients who relapsed from immunotherapies (FIG. 17). These patients would be good candidates for adding MCL1 inhibitors to target MDSCs to improve the efficacy of immunotherapies. For human immune cells, MCL1 inhibitors did not affect T cell proliferation at the concentrations that are likely be physiologically relevant, further illustrating the therapeutic of MCL1 inhibition (FIG. 18).

### CONCLUSION

While multiple embodiments are disclosed, still other embodiments of the present invention will become apparent to those skilled in the art from the detailed description. The invention is capable of modifications in various obvious aspects, all without departing from the spirit and scope of the present invention. Accordingly, the detailed description is to be regarded as illustrative in nature and not restrictive.

All references disclosed herein, whether patent or non-patent, are hereby incorporated by reference as if each was included at its citation, in its entirety. In case of conflict between reference and specification, the present specification, including definitions, will control.

Although the present disclosure has been described with a certain degree of particularity, it is understood the disclosure has been made by way of example, and changes in detail or structure may be made without departing from the spirit of the disclosure as defined in the appended claims.

We claim:

1. A method for enhancing immune function in a patient in need thereof, the method comprising:
administering to the patient a therapeutically effective dose of a first therapeutic composition comprising at least one immunoglobulin;
administering to the patient a therapeutically effective dose of a second therapeutic compound comprising a molecule with affinity for BCL2 family members;
allowing the second therapeutic compound to reduce a size or activity of a population of immunosuppressive cells and increase a size or activity of a population of immunostimulatory cells; and thereby
enhancing immune function in the patient.

2. The method of claim 1, wherein the first therapeutic composition is a checkpoint inhibitor.

3. The method of claim 1 or claim 2, wherein the first population of immunosuppressive cells comprises one or more myeloid-derived suppressor cells.

4. The method of any of claims 1-3, wherein the second population of immunostimulatory cells comprises one or more CD8+ T cells.

5. The method of any of claims 1-4, wherein the patient is a mammal or human.

6. The method of claim 5, wherein the human has a condition selected from a solid tumor, melanoma, virally-induced cancer, or viral infection.

7. A method of inducing solid tumor reduction and/or elimination, comprising:
administering a therapeutic dose of a BCL2 family member inhibitor to inhibit immunosuppression of T cells; and
administering an immunotherapeutic compound to increase immune activation within the solid tumor and induce solid tumor reduction and/or elimination;
wherein the BCL2 family member inhibitor enhances the efficacy of the immunotherapeutic compound.

8. The method of claim 7, wherein the BCL2 family member inhibitor is an MCL1 inhibitor.

9. The method of claim 8, wherein the MCL1 inhibitor inhibits immunosuppression of CD8+ T cells to facilitate solid tumor reduction and/or elimination.

10. The method of any of claims 7-9, wherein the BCL2 family member inhibitor inhibits immunosuppression of T cells by targeting myeloid-derived suppressor cells.

11. The method of any of claims 7-10, wherein the solid tumor is melanoma.

12. A method of improving vaccine function, comprising:
introducing a therapeutically effective dose of a BH3 mimetic to a patient in need of vaccination treatment to reduce or eliminate suppression of the patient's immune system;
vaccinating the patient with a vaccine treatment to produce antibodies and/or T cells having improved immune function due to the therapeutic effect of the BH3 mimetic;
wherein the BH3 mimetic improves the immune system response to the vaccine treatment.

13. A method of treating a subject resistant to or relapsed from an immunotherapy, comprising:
administering a therapeutic compound targeting pro-survival BCL-2 family members to inhibit immunosuppression of T cells; and
administering an immunotherapy to increase T cell recognition of tumor cells;
wherein the immunotherapy blocks the interaction of checkpoint proteins interfering with T cell recognition of tumor cells; and
wherein the immunotherapy when administered by itself, without the therapeutic compound, is insufficient to affect tumor cells.

14. The method of claim 13, wherein the therapeutic compound is an MCL inhibitor.

15. The method of claim 13 or claim 14, wherein the immunotherapy is anti-PD1.

16. The method of any of claims 1-15, wherein the therapeutic compound with affinity for BCL2 family members, is one or more of a MCL inhibitor, BH3 mimetic, S63845, or S64315.

17. A method for treating melanoma in a patient in need thereof, the method comprising:
administering to the patient a therapeutically effective dose of a first therapeutic composition comprising a first BH3 mimetic compound;
administering to the patient a therapeutically effective dose of a second therapeutic composition comprising a second BH3 mimetic compound;
allowing the first and second therapeutic compounds to act synergistically to induce death of melanoma cells; and thereby
effectively treating advanced melanoma in the patient.

18. The method of claim 17, wherein the first therapeutic composition comprises a molecule with BCL2 inhibitory activity.

19. The method of claim 17 or 18, wherein the first therapeutic composition comprises ABT-199/venetoclax.

20. The method of any of claims 17-19, wherein the second therapeutic composition is a molecule with MCL1 inhibitory activity.

21. The method of any of claims 17-20, wherein the second therapeutic composition comprises S63845 or S64315/MIK665.

22. The method of any of claims 17-21, wherein the melanoma does not include BRAF-V600E/K mutations.

23. The method of any of claims 17-22, wherein induction of death is via programmed cell death.

24. The method of any of claims 17-23, wherein the patient is a mammal.

25. The method of any of claims 17-24, wherein the patient is a human.

26. The method of any of claims 17-24, wherein the patient is a pet.

27. The method of claim 26, wherein the patient is refractory to or ineligible for treatment by immune checkpoint blockade.

28. The method of any of claims 17-27, wherein the melanoma is selected from cutaneous, or a subtype selected from uveal, mucosal, and acral.

29. A method for treating a melanoma cell, the method comprising: contacting the cell with a first BH3 mimetic compound; contacting the cell with a second BH3 mimetic compound; thereby treating the cell.

30. The method of claim 29, wherein the first BH3 mimetic compound is a molecule with MCL1 inhibitory activity.

31. The method of any of claim 29 or 30, wherein the first BH3 mimetic compound is S63845 or S64315/MIK665.

32. The method of any of claims 29-31, wherein the second BH3 mimetic compound is a molecule with BCL2 inhibitory activity.

33. The method of any of claims 29-32, wherein the second BH3 mimetic compound is ABT-199/venetoclax.

34. The method of any of claims 29-33, wherein the cell is a human cell selected from a melanoma cell and a melanoma initiating cell.

35. The method of any of claims 29-34, wherein the cell is a melanoma cell that does not include BRAF-V600E/K mutations.

36. The method of any of claims 29-35, wherein treating slows growth of the melanoma cell.

37. The method of any of claims 29-35, wherein treating induces death of the melanoma cell.

38. The method of any of claims 29-35, wherein treating induces programmed cell death.

39. A method of treating melanoma in a patient in need thereof, comprising:
administering to the patient a therapeutically effective dose of a BH3 mimetic compound, wherein the compound inhibits MCL1, and wherein the melanoma is selected from uveal and mucosal subtypes.

40. The method of claim 39, wherein the melanoma is uveal melanoma.

41. A method of selecting a treatment therapy for subject in need thereof, comprising:
obtaining a sample of malignant tissue from the subject;
identifying at least one myeloid derived suppressor cell (MDSC) in the sample; assaying the level of MCL1 expression in the MDSC; and
selecting a therapy for the subject, wherein the therapy includes a MCL1 inhibitory compound when the MDSC of the subject expresses high levels of MCL1.
